(19) United States
(12) Patent Application Publication
(10) Pub. No.: US 2025/0115928 A1
(43) Pub. Date: Apr. 10, 2025

(54) Recombinant Orthopox Viral Vector Encoding Immunostimulatory Proteins for Cancer Treatment

(71) Applicant: Nouscom AG, Basel (CH)

(72) Inventors: Gabriella COTUGNO, Naples (IT); Elisa SCARSELLI, Rome (IT); Anna MORENA D’ALISE, Rome (IT); Armin LAHM, Rome (IT); Guido LEONI, Rome (IT)

(21) Appl. No.: 18/729,271

(22) PCT Filed: Jan. 17, 2023
(86) PCT No.: PCT/EP2023/050917

§ 371 Date: Jul. 16, 2024

(30) Foreign Application Priority Data
Jan. 17, 2022 (EP) 22151830.1

(51) Int. Cl. C12N 15/86; A61K 38/20; A61K 39/395; A61K 48/00; A61P 35/00; C07K 14/54

(52) U.S. Cl. CPC C12N 15/86; A61K 38/208; A61K 39/39558; A61K 48/005; A61P 35/00; C07K 14/5434; C12N 2710/24143

(57) ABSTRACT

The present invention relates to a recombinant Orthopox viral vector comprising, in operable linkage a first promoter comprising or consisting of (i) at least one viral early promoter element and optionally at least one viral late promoter element; or (ii) at least one viral late promoter element and at least three viral early promoter elements, and b) a first nucleic acid sequence encoding at least one immunostimulatory protein. The present invention also relates to cells comprising the recombinant Orthopox viral vectors and to compositions comprising the recombinant Orthopox viral vector or the cells; and optionally a further recombinant viral vector comprising a nucleic acid sequence encoding a check-point inhibitor, a nucleic acid encoding a checkpoint inhibitor or a checkpoint inhibitor. Moreover, the present invention provides the recombinant Orthopox viral vectors, and cells and compositions comprising the same for use in medicine, in particular for use in treating, ameliorating or preventing cancer.

### FIELD OF THE INVENTION

The present invention relates to a recombinant Orthopox viral vector comprising, in operable link-age a first promoter comprising or consisting of (i) at least one viral early promoter element and optionally at least on viral late promoter element; or (ii) at least one viral late promoter element and at least three viral early promoter elements, and b) a first nucleic acid sequence encoding at least one immunostimulatory protein. The present invention also relates to cells comprising the recombinant Orthopox viral vectors and to compositions comprising the recombinant Orthopox viral vector or the cells; and optionally a further recombinant viral vector comprising a nucleic acid sequence encoding a checkpoint inhibitor, a nucleic acid encoding a checkpoint inhibitor or a checkpoint inhibitor. Moreover, the present invention provides the recombinant Orthopox viral vectors, and cells and compositions comprising the same for use in medicine, in particular for use in treating, ameliorating or preventing cancer

### BACKGROUND OF THE INVENTION

Currently, several efforts are made in the field of cancer treatment to overcome the hostile Tumour Microenvironment (TME) that limits the function of anti-tumour T cells.

Macrophages are white blood cells of the mononuclear phagocyte immune system and play an important role in anti-infective immunity, the maintenance of tissue homeostasis, and the protection of the body through the function of internalizing and digesting foreign substances and thus, removing harmful matter, including cellular debris and tumour cells from the body (Zhou et al., Front. Oncol. 2020, Yona S. and Gordon S., Front Immunol. 2015). Further functions of macrophages include mediation of the innate immunity, initiation of the adaptive immunity, secretion of immunomodulatory substances, such as various cytokines or chemokines, and activation of the complement system, which may lead to inflammation. However, macrophage function can be modified and subverted in a harmful manner in the presence of chemokines, cytokines as well as an array of other factors (e.g. CSF-1, CCL2, VEGF, TGFβ, etc.) that are secreted by tumour cells. As such, they are recruited to the tumour microenvironment and become so called tumour-associated macrophages (TAMs) (Watkins et al., J. Immunol. 2021). Tumour-associated macrophages play a major role in tumour progression at different levels, e.g. by promoting tumour growth and metastasis, promoting genetic instability, releasing proteases and other molecules to remodel the extracellular matrix, promoting neo-angiogenesis, and secreting immune-suppressive mediators (Mantovani et al., Nature Reviews Clinical Oncology 14, 2017; Anfrey et al., Cells 2020). TAMs are known to polarize to either M1, which represents anti-tumour activity, or M2, which leads to cancer progression, and are described as M1-like or M2-like macrophages (Murray, P. J. et al., Immunity 41, 14-20, 2014).

Therefore, among strategies to overcome the hostile TME, several efforts are made in the development of strategies to target TAMs, such as inhibition of macrophage recruitment by blocking tumour-derived factors (TDFs) via antibodies (e.g. Pexidarnitib, AMG820 mAb, Carlumab, Emactuzumab, etc.), depletion of TAMs by radiotherapy and/or TDF inhibitory drugs (e.g. PLX3397, trabectedin, etc.), or reprogramming of TAMs from an immune-suppressive M2-like phenotype to a more inflammatory Ml-like state by targeting Toll-Like Receptors (e.g. Imiquimod, Resiquimod, etc.), RNA delivery (e.g. small interfering RNA, microRNA, etc.), or antibodies (e.g. HU5F9-G4 mAb, CP-870,893, APX005M) (Anfrey et al., Cells 2020).

However, in spite of the various TAM-reprogramming approaches, many of them are associated with lack of treatment selectivity and systemic toxicities. As such, there is a high demand for a safe and selective way to shift the tumour microenvironment from an immunosuppressive to an immunostimulatory state.

Here, a Vector Aided Microenvironment Programming (VAMP) strategy has been developed to locally deliver therapeutic proteins and effectively shift the TME from an immunosuppressive to an immunostimulatory state without associated systemic toxicity. Said strategy relates to the provision of a recombinant Orthopox viral vector capable of effective expression of an immunostimulatory therapeutic molecule in the TME under control of promoters that allow for high selective production of the therapeutic protein in the TME and activation of TAMs. Said promoters contain one or more early elements motifs to provide improved expression of proteins of interest, in the target cells, in particular in TAMs. The recombinant viral vectors provide inter alia the following advantages: i) controlled and reproducible delivery of the proteins of interest, in particular immunostimulatory proteins to tumours via infection of normal cells infiltrating tumour ii) efficient reprogramming of M2-like macrophages into MI-like macrophages, iii) limited systemic toxicity, iv) high efficacy in tumours resistant to checkpoint inhibitor (CPI) treatment, and v) significant tumour shrinkage even at very low doses.

### SUMMARY OF THE INVENTION

In a first aspect, the present invention relates to a recombinant Orthopox viral vector comprising, in operable linkage:
a) a first promoter comprising or consisting of:
   (i) at least one viral early promoter element, wherein the viral early promoter element comprises or consists of the nucleic acid sequence AAN¹N²AN³TGAAN⁴N⁵N⁶N⁷N⁸A (SEQ ID NO: 1), wherein each of N¹, N², N⁴, N⁵ and N⁶ is independently selected from A or T, N³ is selected from C, G or T N⁷ is selected from C and A and N⁸ is selected from A, C and T and optionally at least one viral late promoter element; or
   (ii) at least one viral late promoter element and at least three viral early promoter elements, and
b) a first nucleic acid sequence encoding at least one immunostimulatory protein.

In a second aspect, the present invention provides a cell comprising the recombinant viral Orthopox viral vector of the present invention.

In a third aspect, the present invention relates to a composition comprising: a) the recombinant Orthopox viral vector of the first aspect of the invention or the cell of the second aspect of the invention; and b) a pharmaceutically acceptable carrier, and optionally c) a recombinant viral vector comprising a nucleic acid sequence encoding a checkpoint inhibitor, a nucleic acid encoding a checkpoint inhibitor or a checkpoint inhibitor.

In a fourth aspect, the present invention provides the recombinant Orthopox viral vector of the first aspect of the present invention, the cell of the second aspect of the present invention, or the composition of the third aspect of the present invention for use in medicine.

In a fifth aspect, the present invention relates to the recombinant Orthopox viral vector of the first aspect of the present invention, the cell of the second aspect of the present invention, or the composition of the third aspect of the present invention for use in treating or preventing cancer.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents (including all patents, patent applications, scientific publications, manufacturer's specifications, instructions etc.) are cited throughout the text of this specification. Nothing herein is to be construed as an admission that the invention is not entitled to antedate such disclosure by virtue of prior invention.

### Definitions

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

The term “nucleic acid” refers in the context of the present invention to single or double-stranded oligo-or polymers of deoxyribonucleotide or ribonucleotide bases or both. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers. In the context of the present invention, the term nucleic acid includes but is not limited to ribonucleic acid (RNA) and deoxyribonucleic acid (DNA) molecules but also includes synthetic forms of nucleic acids comprising other linkages (e.g., peptide nucleic acids as described in Nielsen et al. (Science 254:1497-1500, 1991). Typically, nucleic acids are single-or double-stranded molecules and are composed of naturally occurring nucleotides. The depiction of a single strand of a nucleic acid also defines (at least partially) the sequence of the complementary strand. The nucleic acid may be single or double stranded or may contain portions of both double and single stranded sequences. Exemplified, double-stranded nucleic acid molecules can have 3- or 5-overhangs and as such are not required or assumed to be completely double-stranded over their entire length. The nucleic acid may be obtained by biological, biochemical or chemical synthesis methods or any of the methods known in the art, including but not limited to methods of amplification, and reverse transcription of RNA. The term nucleic acid comprises chromosomes or chromosomal segments, vectors (e.g., expression vectors), expression cassettes, naked DNA or RNA polymer, primers, probes, cDNA, genomic DNA, recombinant DNA, cRNA, mRNA, tRNA, microRNA (miRNA) or small interfering RNA (siRNA). A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

An “isolated nucleic acid” molecule is one that is separated from other nucleic acid molecules that are present in the natural source of the nucleic acid. For example, with regards to genomic DNA, the term “isolated” includes nucleic acid molecules that are separated from the chromosome with which the genomic DNA is naturally associated. Preferably, an isolated nucleic acid is free of sequences that naturally flank the nucleic acid (i.e., sequences located at the 5′ and 3′ ends of the nucleic acid) in the genomic DNA of the organism from which the nucleic acid is derived. Moreover, an “isolated nucleic acid molecule, such as a cDNA molecule, can be substantially free of other cellular material, or culture medium when produced by recombinant techniques, or substantially free of chemical precursors or other chemicals when chemically synthesized.

The term “gene” in the context of the present invention refers to an assembly of nucleotides that encode an RNA transcript or a polypeptide, and includes cDNA and genomic DNA nucleic acids “Gene” also refers to a nucleic acid fragment that expresses a specific protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes.

The term “genome” as referred to herein includes chromosomal as well as mitochondrial, chloroplast and viral DNA or RNA.

The terms “vector”, “vector construct” or “recombinant vector” may be used interchangeably in the context of the present invention and refer to a polynucleotide that encodes a protein of interest or a mixture comprising polypeptide(s) and a polynucleotide that encodes a protein of interest, which is capable of being introduced or of introducing proteins and/or nucleic acids comprised therein into a cell. Examples of vectors include but are not limited to plasmids, cosmids, phages, viruses or artificial chromosomes. A vector is used to introduce a gene product of interest, such as e.g. foreign or heterologous DNA into a host cell. Certain vectors are capable of directing the expression of genes to which they are operably linked.

Vectors in the context of the invention may further comprise at least one promoter suitable for driving expression of a gene in a host cell. The term “expression vector” means a vector, plasmid or vehicle designed to enable the expression of an inserted nucleic acid sequence following transformation into the host. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art.

Vectors may contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Vectors may further encompass “expression control sequences” that regulate the expression of the gene of interest. Typically, expression control sequences are polypeptides or polynucleotides such as promoters, enhancers, silencers, insulators, or repressors. In a vector comprising more than one polynucleotide encoding for one or more gene products of interest, the expression may be controlled together or separately by one or more expression control sequences. More specifically, each polynucleotide comprised on the vector may be controlled by a separate expression control sequence or all polynucleotides comprised on the vector may be controlled by a single expression control sequence. Polynucleotides comprised on a single vector controlled by a single expression control sequence may form an open reading frame. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized. Such vectors may comprise regulatory elements, such as a promoter, enhancer, terminator and the like, to cause or direct expression of said polypeptide upon administration to a subject.

Preferably, the vector comprises an expression cassette comprising a promoter and a coding sequence, wherein the expression of the coding sequence is controlled by said promoter.

Preferably, the vector in context of this invention is a viral vector. The term “viral vector” or “virus vector” refers to a nucleic acid vector construct that includes at least one element of viral origin and has the capacity to be packaged into a viral vector particle, and encodes at least an exogenous nucleic acid. The vector and/or particle can be utilized for the purpose of transferring any nucleic acids into cells either in vitro or in vivo. Numerous forms of viral vectors are known in the art. The term “virion” is used to refer to a single infective viral particle. The terms “viral vector”, “viral vector particle” and “viral particle” also refer to a complete virus particle with its DNA or RNA core and protein coat as it exists outside the cell.

Preferably, the viral vectors in the context of the present invention can be used for the infection of cells and cell lines, in particular for the infection of living animals including humans. More preferably, the viral vectors according to the present invention infect antigen presenting cells, and most preferably macro-phages. Typical viral vector may be selected from adenoviruses, poxviruses, alphaviruses, arenaviruses, flaviruses, rhabdoviruses, retroviruses, lentiviruses, herpesviruses, paramyxoviruses, or picornaviruses. Preferably, the viral vector is from a poxvirus, more preferably from a vaccinia virus, and most preferably from a Modified Vaccinia Ankara (MVA) virus.

In general, the recombinant viral vectors of the invention are capable of being packaged into a viral particle. For example, a recombinant Modified Vaccinia Virus vector of the invention is capable of being packaged into a Modified Vaccinia Virus particle. Preferably, the virion or viral particle is attenuated, which means that a virus has the capability of reproductive replication in avian cells, such as in chicken embryo fibroblasts (CEF) or immortalized cell lines derived from avian primary cells like Duck Cairina primary retina cells but no capability of productive replication in human cell lines, such as the human embryo kidney cell line 293, the human cervix adenocarcinoma cell line HeLa, etc.

MVA is related to the vaccinia virus, a member of the genus Orthopoxvirus in the family of Poxviridae and has been generated by more than 570 serial passages in primary chicken embryo fibroblast (CEF) cells (Mayr, A et al., Infection 3, 6-14, 1975). As a consequence of these long-term passages, the host range of the virus was severely restricted leading to the inability of MVA to productively infect many mammalian cells. On the other hand, MVA has shown an excellent safety profile and immunogenicity in the clinic and is well tolerated, highlighting its potential as a safe vector for the development of vaccine and gene therapy candidates. The term “MVA” as used in the present application refers also to any MVA strain known in the prior art. Preferred examples of MVA strains that may be the basis of the recombinant Orthopox viral vectors of the invention are the strain MVA-BN (the nucleic acid sequence of the genome of this strain is accessible in GenBank Accession No.: DQ983238.1), the strain MVA 572 (the nucleic acid sequence of the genome of this strain is accessible in GenBank: Accession No.: DQ983237.1), MVA-I721 (the nucleic acid sequence of the genome of this strain is accessible in GenBank Accession No.: DQ983236.1), Acambis 3000 (GenBank: Accession No.: AY603355.1) or the strain MVATGN33.1 (the nucleic acid sequence of the genome of this strain is accessible in GenBank: Accession No.: EF675191.1).

The term “promoter” refers in the context of this invention to a regulatory region of DNA generally located upstream (towards the 5′ region of the sense strand) of a gene that allows transcription of the gene. The promoter contains specific DNA sequences and response elements that are recognized by proteins known as transcription factors. These factors bind to the promoter sequences, recruiting RNA polymerase, and the enzymes that synthesizes the RNA from the coding region of the gene. The terms “upstream” and “downstream” are terms used to describe the relative orientation between two elements present in a nucleotide sequence or vector. An element that is “upstream” of another is located in a position closer to the 5′ end of the sequence (i.e., closer to the end of the molecule that has a phosphate group attached to the 5′ carbon of the ribose or deoxyribose backbone if the molecule is linear) than the other element. An element is said to be “downstream” when it is located in a position closer to the 3′ end of the sequence (i.e., the end of the molecule that has a hydroxyl group attached to the 3′ carbon of the ribose or deoxyribose backbone in the linear molecule) when compared to the other element.

The term “late promoter element” in context of the present invention refers to a nucleic acid sequence present in the genome of a virus that drives expression of viral genes during the late stage of infection of a cell by the virus. The term “later promoter element” also comprises variants of the naturally occurring viral late promoter elements. Preferably such variants have at least a 60%, more preferably at least 70%, more preferably at least 80%, more preferably at least 90%, and even more preferably at least 95% nucleic acid sequence identity to a naturally occurring viral early promoter element and lead at least to the same level of transcription of a gene under the control of that variant viral late promoter element as the natural occurring viral late promoter element. Transcription levels of a gene under control of such a promoter can be determined using art known methods including in particular quantitative PCR (qPCR) of cDNA generated from the RNA isolated form a cell infected with the viral vector.

The term “early promoter element” in context of the present invention refers to a nucleic acid sequence present in the genome of a virus that drives expression of viral genes during the early stage of infection of a cell by the virus. The term “early promoter element” also comprises variants of the naturally occurring viral early promoter elements, e.g. variant viral early promoter elements. Preferably such variants have at least a 60%, more preferably at least 70%, more preferably at least 80%, more preferably at least 90%, and even more preferably at least 95% nucleic acid sequence identity to a naturally occurring viral early promoter element and lead at least to the same level of transcription of a gene under the control of that variant viral early promoter element as the natural occurring viral early promoter element. Transcription levels of a gene under control of such a promoter can be determined using art known methods including in particular quantitative PCR (qPCR) of cDNA generated from the RNA isolated form a cell infected with the viral vector.

Preferably, the promoter in the context of the present invention is suitable to for the expression of heterologous genes in poxviruses. Poxviruses control their gene expression at the level of transcription through a cascade-like mechanism involving three major classes of genes: early, intermediate and late, with the latter two classes being expressed after genome replication. As such, promoters with an activity for any of the gene classes are preferred. Promoters with both early and late activity are commonly used to direct the expression of foreign antigens in poxvirus vectors which are utilized as vaccine vectors and injected preferably intramuscularly to ensure that adequate expression levels are present at the appropriate time for induction of strong immune responses. Such promoters include, but are not limited to native poxvirus promoters that drive the expression of a viral protein, such as p7.5k 30k and 40k promoters. In addition, synthetic promoters may employ multiply early and late elements. For example, the promoter pHyb has been shown to drive the expression of antigens earlier during infection when compared to the PrS and p7.5k promoters and also to induce stronger CD8 T cell responses after repeat vaccinations. Thus, the term “early promoter” in context of the present invention refers to promoters that are active in poxviruses virus or cells infected with a poxvirus in the early phase, before viral DNA replication has occurred. Accordingly, the term “late promoter” refers to any promoters that are active after DNA replication has taken place. As such, the term “early-late promoter” in context of the present specification refers to a promoter which is active in both, the timeframe of an early as well as a late promoter. The promoters in the context of the present invention are preferably synthetic and comprise at least one poxvirus early element, more preferably one late and at least three early poxvirus promoter elements.

The term “in operable linkage” or “operably linked” in the context of this invention refers to an arrangement of two or more components, wherein the components are in a relationship permitting them to function in a coordinated manner. By way of illustration, a promoter is operably linked to a coding sequence if the promoter drives transcription of the coding sequence. Aspects of the transcription process include, but are not limited to, initiation, elongation, attenuation and termination.

The term “therapeutic protein” in context of the present invention, refers to genetically engineered proteins that are widely used in medicine. Based on their pharmacological activity, they can be divided into five groups: (a) replacing a protein that is deficient or abnormal; (b) augmenting an existing pathway; (c) providing a novel function or activity; (d) interfering with a molecule or organism; and (c) delivering other compounds or proteins, such as a radionuclide, cytotoxic drug, or effector protein. They can also be classified based on their molecular types, (e.g. antibody-based drugs, Fc fusion proteins, anticoagulants, blood factors, bone morphogenetic proteins, engineered protein scaffolds, enzymes, growth factors, hormones, interferons, interleukins, and thrombolytics, etc.) or on their molecular mechanism of activity as (a) binding non-covalently to target, e.g., mAbs; (b) affecting covalent bonds, e.g., enzymes; and (c) exerting activity without specific interactions, e.g., serum albumin (Dimitrov, D. S., Methods Mol. Biol. 2012).

The terms “immunostimulatory molecule”, “immunostimulant” or “immunostimulator” can be used interchangeably and refer in the context of the present invention to a substance that induce the activation or increase the activity of any component of immune system, thereby stimulating the immune system. There are a multitude of molecules known to the skilled person that are capable of stimulating the immune system. In this regard, of particular interest in context with the present invention are inflammatory or pro-inflammatory molecules.

The term “inflammatory molecule” or “pro-inflammatory molecule” as used herein refers to a molecule able to shift the tumour microenvironment (TME) from an immunosuppressive to an immunostimulatory state. Immunostimulatory molecules can be secreted from immune cells, such as helper T cells, macrophages, astrocytes, monocytes, etc., that promote inflammation. In particular, the term “inflammatory molecule” refers to cytokines.

The term “cytokine” or “(pro-)inflammatory cytokine” in context of the present invention refers to polypeptides produced throughout the body predominantly by activated macrophages. These molecules have an important role in many physiological responses and have diverse effects, including autocrine (act on the cells that secrete them), paracrine (act on nearby cells), endocrine (act on distant cells), and juxtacrine actions (transmitted via oligosaccharide, lipid or protein components of closely apposed cell membranes). The classic role of these molecules is related to the regulation of immune and inflammatory processes (Navarro-González et al., Nat. Rev. Nephrol. 7, 327-340, 2011). Typical cytokines involved in inflammation can be classified as interleukins, e.g. IL-1, IL-6, IL-12, IL-15; interferons, e.g. IFNα, IFNβ, IFNγ; tumour necrosis factors, e.g. TNFα, TNFβ; chemokines, e.g. CC, CXC, CX3 chemokines; colony stimulating factors, e.g. GM-CSF, G-CSF, M-CSF, IL-3; growth factors, e.g. EPO, TPO, EGF, FGF, PDGF, BDNF, VGF, TGFβ; adhesion molecules, e.g. ICAM, VCAM; enzymes, e.g. phospholipase A; complement related molecules, e.g. C3, C5; and other molecules, e.g. PAI-1, MIF, pentraxin, SAA, lactoferrin, procalcitonin, LCN2.

The term “interleukin” or “IL” as used in the context of the present invention, refers to a type of cytokines that regulate inflammatory and immune responses. The sources of interleukins are manifold and not only include white blood cells, but also nearly all lymphocytes and tumour cells. Interleukins are produced by a variety of cells and also act on many cells, constituting a complex regulatory network. Generally, the interleukin families have three main functions: i) activating and regulating immune cells, ii) transmitting information in a variety of cells, and iii) participating in inflammatory response. Presently, there is a total of 38 identified interleukins which are denominated as IL-1 to IL-38. Depending on the differences in their molecular structures and their receptors, they can be further divided into the IL-1, IL-6, IL-10, IL-12 and IL-17 families, interleukin members in the chemokine family, and unclassified interleukins. Each of the interleukin families include several members of interleukins.

In context with the present invention, the term “interleukin 12” or “IL-12”, refers to a heterodimeric cytokine that belongs to the interleukin 12 (IL-12) family, which is comprised of the four members IL-12, IL-23, IL-27 and IL-35. This family plays a crucial role in shaping immune responses during antigen presentation and influences cell-fate decision of differentiating naïve T cells. In addition, the IL-12 family regulates cellular pathways required for proper functioning of the immune system, with some members activating pro-inflammatory responses that confer protection against infection while others restrain unbridled immune responses that cause autoimmune diseases (Sun et al., Cytokine 2015, 75(2): 249-255). IL-12, IL-23 and IL27 are secreted by activated antigen presenting cells (APCs) antigen presentation to naïve T cells while IL-35 is a product of regulatory T and B cells. Each member is composed of an α-subunit with a helical structure, i.e. IL-12p35, IL-23p19 and IL27p28, and a β-subunit, i.e. IL-12p40 and Ebi3, covalently linked by a disulphide bridge.

The term “single chain IL-12” (sc-IL12) in context of the present specification, means an IL-12 which has been engineered to express the IL-12p40 polypeptide fused via linker sequence to the IL-12p35 polypeptide such that the p40/p35 molecule is produced as a single polypeptide chain. The configuration can be in cither order such that the single polypeptide is produced beginning with the p40 polypeptide as the amino-terminal portion (N-terminal) linked to the p35 polypeptide as the carboxyl-terminal portion (C-terminal) in a format designated as “p40-linker-p35”. Conversely, in a scIL-12 construct, the p35 portion can also be the N-terminal portion linked to p40 as the C-terminal portion in a format designated as “p35-linker-p40”. Further possible configurations include also “p40-linker-p35-linker-p40” or “p35-linker-p40-linker-p35”. Secretion of sc-IL 12 into the extracellular space is accomplished by the presence of a signal peptide at the N-terminal end of the sc-IL 12, preferably the signal peptide is from the human IL-12p40 or the human IL-12p35, more preferably from human IL-12p40.

The term “linker” refers in the context of the present invention to a nucleic acid sequence or amino acid sequence which sterically separates two parts or moieties of a complex, e.g. two peptides, polypeptides or proteins, nucleic acids with a particular function, e.g promoter elements. Peptide linker provide flexibility among the two moieties that are linked together. Flexibility is generally increased if the amino acids are small. Typically, such linker comprises or consists of between one and 20 amino acids Accordingly, flexible peptide linkers comprise an increased content of small amino acids, in particular of glycine and/or alanine, and/or hydrophilic amino acids such as serine, threonine, asparagine and glutamines. In the context of the present invention, linkers, e.g. one or more amino acids, inserted between two domains provide sufficient mobility for the domains, for example in single chain constructs. Nucleotides linker between promoter elements have the purpose of spacing the elements to provide promoter binding proteins, e.g. transcriptional activators, with sufficient space to bind to each of the respective promoter elements. About 7 consecutive nucleotides constitute a full turn of a double helix. If two elements are separated by 7 nucleotides they will be located at the same spatial orientation, e.g. the same site of the DNA double helix. Thus, it is preferred that nucleic acid linkers between to nucleotide sequence elements have a length between 5 to 8 nucleotides, like the linker elements inserted between late promoter elements, between early promoter elements and between late and early promoter elements in the promoters of the present invention.

The term “checkpoint inhibitor” or “immune checkpoint inhibitor” (ICI) as referred to in the present invention, refers to drugs. i.e. monoclonal antibodies, that specifically target immune checkpoints and block their function, and are used predominantly in the field of tumour therapy. Several small molecules targeting other immune checkpoints such as LAG3, TIGIT, TIM3, B7H3, CD39, CD73, adenosine A2A receptor, and CD47 are in clinical development. Checkpoint inhibitors work by releasing the inhibitory breaks of T cells, resulting in robust activation of the immune system and productive anti-tumour immune response. ICIs can be classified in three FDA (US Food and Drug Administration) approved drug groups depending on the molecules they are targeting. These drugs include antibodies against cytotoxic T lymphocyte-associated protein 4 (CTLA-4), antibodies that block the inhibitory receptor, programmed cell death 1 (PD-1), on T cells, which interacts with its ligands, PD-L1 and PD-L2, to thwart active T cell responses, and antibodies against PD-L1. An example of an approved CTLA-4 inhibitor is Ipilimumab. Examples of approved PD-1 or PD-L1 inhibitors include Pembrolizumab, Nivolumab, Cemiplimab, Atezolizumab, Avelumab and Durvalumab.

The term “antigen” means a substance that can be recognized by an antibody, B cell or T cell. The term “tumour antigen” or “tumour associated antigen (TAA) as used to in the context of the present invention refers to a protein or polypeptide or an antigenic fragment thereof that is expressed by a tumour cell. Antigenic fragments are typically presented by MHC-I or MHC-II and elicit a T cell response.

The term “macrophage” in the context of the present invention, refers to myeloid immune cells that are found in all tissues and exhibit different phenotypes and great functional diversity. They have important roles in development, homeostasis, tissue repair, immunity and inflammation processes. Broadly, macrophages can be activated into two distinct subsets based on the M1/M2 paradigm, classically activated (M1 macrophages) macrophages or alternatively activated (M2) macrophages. M1 macrophages are polarized in vitro by Th1 cytokines such as colony-stimulating factor (GM-CSF), tumour necrosis factor α (TNF-α) and interferon-γ (IFN-γ) alone or together with lipopolysaccharide (LPS) from bacteria. M1 macrophages express pro-inflammatory cytokines such as interleukin-1β (IL-1β), IL-6, IL-12, IL-23, and TNF-α. In contrast, M2 macrophages are polarized by Th2 cytokines such as IL-4 and IL-13 and produce anti-inflammatory cytokines such as IL-10 and transforming growth factor beta (TGF-β). Macrophages have also the ability to change their polarization in response to different stimuli (Zhang et al., Front. Immunol., 2021).

The term “tumour-associated macrophages (TAMs)” refers to macrophages that participate in the formation of the tumour microenvironment. They are widely present in various tumours and can promote tumour growth, invasion, metastasis and drug resistance.

The term “polarization” in the context of the present specification, designates the phenotypic features and the functional features of the macrophages. The phenotype can be defined through the surface markers expressed by the macrophages. The functionality, can be defined for example based on the nature and the quantity of chemokines and/or cytokines expressed by the macrophages. The macrophages may present different phenotypic and functional features depending on their state, either pro-inflammatory M1-like macrophages or anti-inflammatory M2-like macrophages.

The term “M1 macrophages” in the context of the present application, refer to pro-inflammatory macrophages or classically activated macrophages. They are highly phagocytic and produce large amounts of reactive oxygen and nitrogen species, thereby promoting a Th1 response. They are also defined by the expression of surface markers such as CD68 and CCR7. M1 macrophages secrete high levels of inflammatory cytokines such as IL-12 and IL-23. IL-12 induces the activation and clonal expansion of Th17 cells, which secrete high amounts of IL-17, which contributes to inflammation. These characteristics allow M1 macrophages to control metastasis, suppress tumour growth, and control microbial infections. Moreover, the infiltration and recruitment of M1 macrophages to tumour sites correlates with a better prognosis and higher overall survival rates in patients with solid tumours. After recognition, malignant cells can be destroyed by M1 macrophages through several mechanisms, which include contact-dependent phagocytosis and cytotoxicity (i.e. cytokine release such as TNF-α).

The terms “M1-like macrophages” or “M1-polarized macrophages” in the context of the present application, refer to macrophages that include those polarization states leading to anti-tumour responses and cytotoxicity, such as that induced by GM-CSF.

The term “M2-like macrophages” or “M2-polarized macrophages” as used in this specification, refers to anti-inflammatory macrophages which aid in the process of angiogenesis and tissue repair. They can be characterized by the expression of surface markers such as CD206, PD-LI and CD200R, express scavenger receptors and produce large quantities of IL-10 and other anti-inflammatory cytokines. Expression of IL-10 by M2 macrophages promotes a Th2 response. Th2 cells consequently upregulate the production of IL-4 and IL-3, the latter which stimulates proliferation of all cells in the myeloid lineage (granulocytes, monocytes and dendritic cells), in conjunction with other cytokines (e.g. erythropoietin, granulocyte macrophage colony-stimulating vector (GM-CSF), and IL-6. M2 macrophages exhibit functions that may help tumour progression by allowing blood vessels to feed the malignant cells and thus, promoting their growth. In addition, the presence of M2 macrophages has been linked to the metastatic potential in breast cancer.

The term “amino acid” as used in the context of the present invention refers to to any monomer unit that comprises a substituted or unsubstituted amino group, a substituted or unsubstituted carboxy group, and one or more side chains or groups, or analog of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. As used herein, the term “amino acid” includes the following twenty natural or genetically encoded alpha-amino acids: alanine (Ala or A), arginine (Arg or R), asparaginc (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these are to be interpreted as “any amino acid.” The structures of these twenty natural amino acids are shown in, e.g., Stryer et al., Biochemistry, 5^{th} ed., Freeman and Company (2002).

The terms “sequence identity” or “sequence homology” as referred to in the present specification are interchangeable and are used with regard to polypeptide and nucleotide sequence comparisons. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID NO, if not specifically indicated otherwise. For example, a polypeptide sequence consisting of 200 amino acids compared to a reference 300 amino acid long polypeptide sequence may exhibit a maximum percentage of sequence identity of 66.6% (200/300) while a sequence with a length of 150 amino acids may exhibit a maximum percentage of sequence identity of 50% (150/300). If 15 out of those 150 amino acids are different from the respective amino acids of the 300 amino acid long reference sequence, the level of sequence identity decreases to 45%. The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package. http://hmmer.wustl.edu/) or with the CLUSTAL algorithm (Thompson. J. D., Higgins. D. G. & Gibson. T. J. (1994) Nucleic Acids Res. 22, 4673-80) available e.g. on http://www.ebi.ac.uk/Tools/clustalw/ or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/ or http://www.ebi.ac.uk/Tools/clustalw2/index.html. The grade of sequence identity (sequence matching) may be calculated using e.g. BLAST, BLAT or BlastZ (or BlastX). BLAST protein searches are performed with the BLASTP program, score=50, word length=3. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl. 1: I54-I62) or Markov random fields. Structure based alignments for multiple protein sequences and/or structures using information from sequence database searches, available homologs with 3D structures and user-defined constraints may also be used (Pei J, Grishin NV: PROMALS: towards accurate multiple sequence alignments of distantly related proteins. Bioinformatics 2007, 23:802-808; 3DCoffee@igs: a web server for combining sequences and structures into a multiple sequence alignment. Poirot O, Suhre K, Abergel C, O'Toole E, Notredame C. Nucleic Acids Res. 2004 Jul. 1; 32: W37-40.). When percentages of sequence identity are referred to in the present application, these percentages are calculated in relation to the full length of the longer sequence, if not specifically indicated otherwise.

The term “pharmaceutical composition” as used herein refers to the combination of an active agent with a pharmaceutically acceptable carrier, inert or active, a diluent, and an excipient, making the composition suitable for therapeutic use. In addition, pharmaceutical compositions comprising the conjugate of the present invention can be formulated for oral, parenteral, topical, inhalative, rectal, sublingual, transdermal, subcutaneous or vaginal application routes according to their chemical and physical properties. Pharmaceutical compositions comprise solid, semisolid, liquid, or transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the conjugates of the present invention. Semisolid compositions that can be used in the context of the invention comprise emulsion, suspension, creams, lotions, gels, globules, buccal tablets and suppositories. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides.

The term “pharmaceutically” or “pharmaceutically acceptable” refers in the context of the present invention to molecular entities and compositions that do not lead to an adverse, allergic or other unwanted reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The term “carrier”, in the context of the present invention refers to a diluent, adjuvant, excipient, or vehicle with which the therapeutic agent is administered. Such pharmaceutical carriers can be liquid or solid. Liquid carriers include but are not limited to sterile liquids, such as saline solutions in water and oils, including but not limited to those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

A “pharmaceutically acceptable carrier” in the context of the present invention may also be referred to as “pharmaceutically acceptable diluent” or “pharmaceutically acceptable vehicles” and may include solvents, bulking agents, stabilizing agents, dispersion media, coatings, antibacterial and antifungal agents, isotonic and absorption delaying agents, and the like which are physiologically compatible.

The term “excipient” in the context of the present invention refers to any substance other than the active substance, present in a medicinal product or used in the manufacture of the product. Excipients function as a carrier of the active substance and contribute to product attributes such as stability, biopharmaceutical profile, appearance and patient acceptability. Suitable pharmaceutical excipients include starch, glucose, lactose, sucrose, gelatine, malt, rice flour, chalk, silica gel, sodium stearate, glycerol monostearate, talc, sodium chloride, dried skim milk, glycerol, propylene glycol, water, ethanol and the like.

The term “adjuvant” as referred to in the present invention, means a substance or combination of substances that are added to a vaccine, pharmaceutical composition or medicament to enhance the effectivity of their active components as well as stimulate and enhance the magnitude and durability of an immune response.

The term “effective amount” and “therapeutically effective amount” refers to an amount that may be effective to elicit a desired biological or medical response, including the amount of a compound that, when administered to a subject for treating a disease, is sufficient to affect such treatment for the disease. The effective amount will vary depending on the compound, the disease and its severity, the age, weight etc. of the subject to be treated, which can readily be determined by one skilled in the art. The effective amount can include a range of amounts. A pharmaceutically effective amount includes amounts of an agent which are effective when combined with other agents.

As used herein, “treat”, “treating” or “treatment” of a disease or disorder means accomplishing one or more of the following: (a) reducing the severity of the disorder, (b) limiting or preventing development of symptoms characteristic of the disorder(s) being treated, (c) inhibiting worsening of symptoms characteristic of the disorder(s) being treated, (d) limiting or preventing recurrence of the disorder(s) in subjects that have previously had the disorder(s); and (c) limiting or preventing recurrence of symptoms in subjects that were previously symptomatic for the disorder(s).

As referred to in the present specification, the terms “prevent”, “preventing” or “prevention” of a disease or disorder mean preventing that a disorder occurs in a subject for a certain amount of time. For example, if a compound described herein is administered to a subject with the aim of preventing a disease or disorder, said disease or disorder is prevented from occurring at least on the day of administration and also on one or more days preferably months or years following the day of administration. More specifically in cancer prevention this can apply to the treatment of a pre-cancerous lesions to avoid progression to a cancer.

As used in the context of the present invention, the terms “ameliorate”, “ameliorating” or “amelioration” of a disease or disorder refer to any indication of success in the treatment of said disease or disorder, including any objective or subjective parameter such as abatement, remission or diminishing of symptoms or an improvement in a subject's physical well-being. Amelioration of symptoms can be based on objective or subjective parameters, including the results of a physical examination or evaluation.

According to the present invention, the term “subject” refers to an animal, including a human being. The term “animal” includes any animal, such as, but not limited to primates including humans, gorillas and monkeys; rodents, such as mice and rats; fowl, such as chickens; ruminants, such as goats, cows, deer, sheep; ovine, and other animals including pigs, horses, cats, dogs, and rabbits.

### Embodiments

In the work leading to the present invention, it was surprisingly found that recombinant Orthopox viral vectors of the present invention allow a reproducible and controlled delivery of an immunostimulant to tumours via infection of normal cells infiltrating tumour. In addition, the vectors according to the present invention showed a surprisingly efficient and high expression of the encoded immunostimulant while maintaining limited systemic toxicity due to the controlled delivery of the immunostimulant only to the tumours. Moreover, the vectors exhibit efficient reprogramming of M2-like macrophages into M1-like macrophages, high efficacy in tumours resistant to checkpoint inhibitor (CPI) treatment, and significant tumour shrinkage even at very low doses.

Based on these results, the present invention provides in a first aspect a recombinant Orthopox viral vector comprising, in operable linkage:
a) a first promoter comprising or consisting of:
   (i) at least one viral early promoter element, wherein the viral early promoter element comprises or consists of the nucleic acid sequence AAN¹N²AN³TGAAN⁴N⁵N⁶N⁷N⁸A (SEQ ID NO: 1), wherein each of N¹, N², N⁴, N⁵ and No is independently selected from A or T (preferably A), N³ is selected from C, G or T (preferably T), N⁷ is selected from C and A (preferably C) and N⁸ is selected from A, C and T (preferably T), and optionally at least one viral late promoter element; or
   (ii) at least one viral late promoter element and at least three viral early promoter elements, and
b) a first nucleic acid sequence encoding at least one immunostimulatory protein.

According to some embodiments, the recombinant Orthopox viral vector is an expression vector capable of mediating the expression of an inserted nucleic acid in a host cell. In this case, the expression vectors of the present invention comprise an inserted nucleic acid encoding at least one therapeutic protein under the control of an operably linked promoter. It is to be understood by a skilled person that the expression vector may also comprise further elements necessary for expression, such as an origin of replication, a translation initiation sequence, e.g. a ribosomal binding site and a start codon, a termination codon, and a transcription termination sequence.

According to another embodiment, the recombinant Orthopox viral vector comprises an expression cassette. In this regard, the expression cassettes in the recombinant Orthopox viral vector of the present invention comprises a promoter, a gene of interest, e.g. an immunostimulatory molecule and/or a tumour antigen. The vector may include sequences flanking the expression cassette that include sequences homologous to eukaryotic genomic sequences, such as mammalian genomic sequences, or viral genomic sequences.

According to a further embodiment, the recombinant Orthopox viral vector of the present invention is an infectious virion or viral particle comprising the expression cassette. These virions or viral particles are capable of infecting a variety of cells and cell lines, in particular capable of infecting living animals, including humans. Preferably, the virions or viral particles are efficient in infecting antigen presenting cells (APCs), such as dendritic cells, macrophages, or B cells. More preferably, the virions or viral particles infect macrophages. Even more preferably, the virions or viral particles infect healthy macrophages. The virions or viral particles may also infect tumour-associated macrophages.

In some embodiments, the infection of cells occurs by binding of the virion or virus particle to cell surface molecules, preferably receptors. More preferably, the receptors are class A scavenger receptors. Typical class A scavenger receptors include Scavenger receptors type 1 (SR-A1, also known as SCARA1 or MSR1), SCARA2 (also known as MARCO or SR-A6), SCARA 3 (also known as MSRL1, APC7 or SR-A3), SCARA4 (also known as COLEC12 or SR-A4) and SCARA5 (also known as TESR or SR-A5). In preferred embodiments the infection of cells occurs via SCARA2, which is also known as MARCO (macrophage receptor with collagenous structure). Preferably, the virions or viral particles in context of the present invention, bind directly to SCARA2 (MARCO).

It is further preferred that the Orthopox viral vectors in context of the present invention are infectious while exhibiting impaired replication of the virus in the cells, thus providing a natural limitation on infections with the virus.

According to one embodiment, the recombinant Orthopox virus vector is based on a virus of the species selected from the group consisting of Orthopoxvirus variola, Orthopoxvirus vaccinia, Orthopoxvirus simiae, Orthopoxvirus bovis, Orthopoxvirus muris, Orthopoxvirus cameli, Raccoonpox virus or Taterapox virus. Preferably, the Orthopox virus is of the species Orthopoxvirus vaccinia. According to a more preferred embodiment, the Orthopox virus of the species Orthopoxvirus vaccinia is of the subspecies Modified Vaccinia Ankara Virus (MVA).

Recombinant viral vectors based on modified Vaccinia Ankara viruses are of particular interest in context of the present invention since the present inventors have shown that they primarily infect antigen presenting cells including macrophages and are described to be avirulent as a consequence of the genomic sequence deletion during the long-term passaging. In addition, MVA is very promising for the expression of heterologous genes due to its improved safety profile and their genetic plasticity, which allows large amounts of foreign DNA to be incorporated without loss of infectivity or reduction of gene expression.

The recombinant Orthopox viral vectors of the present invention further have the utility for facilitating the expression of a broad number of exogenous nucleic acid sequences. The recombinant Orthopox viral vectors of the present invention are used in an immunomodulatory therapeutic approach. Accordingly, the Orthopox viral vectors comprise a first nucleic acid sequence encoding at least one immunostimulatory molecule.

In one embodiment, the recombinant Orthopox viral vectors of the present invention may be for use in vitro and in vivo. Classes of genes contemplated for expression with the vectors of the present invention include immunostimulatory or immunomodulatory proteins.

According to one embodiment, the immunostimulatory protein is a pro-inflammatory protein. According to a preferred embodiment, the pro-inflammatory protein is a cytokine. According to an even more preferred embodiment, the cytokine is an interleukin.

In a particularly preferred embodiment, the interleukin is of the interleukin 12 family. The interleukin 12 (IL-12) family comprises of the four members, IL-12, IL-23, IL-27 and IL-35. According to the most preferred embodiment, the interleukin is an interleukin 12.

IL-12 is secreted by a variety of hematopoietic cell types, such as dendritic cells and macrophages. IL-12 is also a strong pro-inflammatory cytokine that leads to the secretion of other cytokines including tumour necrosis factor-alpha (TNF-α) which, combined with IFN-γ, is a prerequisite for the development of CD4⁺ cytotoxic T lymphocytes (CTL). IL-12 has also been reported to induce the repolarization of tumour-associated macrophages.

IL-12 is comprised of the IL-12p35 and IL12-p40 subunits, which need to be expressed simultaneously to produce the biologically active dimer form IL-12p70. This may either be attained by expressing both subunits as one transcript with an intervening Internal Ribosome Entry Site (IRES) or with a sequence that encodes a self-cleaving amino acid sequence or with a linker that connects the subunits generating a single-chain IL-12 or by expressing two separate transgenes either from the same promoter or two promoters. Suitable amino acid sequences are murine IL-12 p40 subunit with an amino acid sequence according to SEQ ID NO: 31, murine IL-12 p35 subunit with an amino acid sequence according to SEQ ID NO: 32 (without N-terminal signal sequence), human IL-12 p40 subunit with an amino acid sequence according to SEQ ID NO: 33, human IL-12 p35 subunit with an amino acid sequence according to SEQ ID NO 34 (without N-terminal signal sequence). Preferably, the IL-12 is human single chain IL-12 (sc-hIL12), preferably with an amino acid sequence according to SEQ ID NO: 38 to 40, preferably SEQ ID NO: 38.

According to some embodiments, the recombinant Orthopox viral vectors promote IL12 production by macrophages. In some embodiments, the recombinant Orthopox viral vectors induce conversion or repolarization of macrophages into other functional phenotypes. According to another embodiment, the macrophages are driven (repolarized) towards a proinflammatory M1 phenotype and/or away from an anti-inflammatory M2 phenotype.

According to another embodiment, the recombinant Orthopox viral vector further comprises a second nucleic acid sequence encoding at least one tumour antigen or an antigenic fragment thereof.

The nucleic acid sequences to be expressed are placed in operable linkage of to a promoter.

According to one embodiment, the two transgenes are linked to one promoter and separated by an intervening Internal Ribosome Entry Site (IRES) or separated with a sequence that encodes a self-cleaving amino acid sequence.

According to another embodiment, the two transgenes are linked to two independent promoters that may be the same or preferably be different promoters.

Preferably the different nucleic acid elements comprised in the Orthopox viral vector of the invention are arranged in 5′ to 3′ direction (with reference to the coding nucleotide sequence):
[late promoter element]ₘ-[early promoter element]ₙ-nucleic acid sequence encoding at least one immunostimulatory protein.
wherein m is 0 to 5, i.e. 1, 2, 3, 4, or 5 and n is between 1 to 10, i.e. 1, 2, 3, 4, 5, 6, 7, 8, 9, or 10, preferably 2 to 8, more preferably 4 to 6. In a particular embodiment a nucleic acid linker is present between two or all early promoter elements and/or the early promoter element(s) and the late promoter element. It is preferred that m is 1 or 2 more preferably 1, i.e. that one or two late promoter element(s) are located upstream (or 5′ prime) of 1, 2, 3, 4, 5, 6, 7, 8, 9, or 10, preferably 2 to 8, more preferably 4 to 6 early promoter elements.

In the Orthopox viral vector of the first aspect of the invention the viral early promoter element is one that is naturally found in an Orthopox virus. Preferably, the viral early promoter element is one that is naturally found in virus of the species selected from the group consisting of Orthopoxvirus variola, Orthopoxvirus vaccinia, Orthopoxvirus simiae, Orthopoxvirus bovis, Orthopoxvirus muris, Orthopoxvirus cameli, Raccoonpox virus or Taterapox virus. Preferably, the viral early promoter element is one that is naturally found in virus of the species Orthopoxvirus vaccinia. According to a more preferred embodiment the viral early promoter element is one that is naturally found in a Modified Vaccinia Ankara Virus (MVA). Alternatively, the viral early promoter element is a structural variant of such a promoter element. Preferably such variants have at least a 60%, more preferably at least 70%, more preferably at least 80%, more preferably at least 90%, and even more preferably at least 95% nucleic acid sequence identity to a naturally occurring viral early promoter element and lead at least to the same level of transcription of a gene under the control of that variant viral early promoter element as the natural occurring viral early promoter element. Transcription levels of a gene under control of such a promoter can be determined using art known methods including in particular quantitative PCR (qPCR) of cDNA generated from the RNA isolated form a cell infected with the viral vector.

The Orthopox viral vector of the first aspect of the invention the viral late promoter element is one that is naturally found in an Orthopox virus. Preferably, the viral late promoter element is one that is naturally found in virus of the species selected from the group consisting of Orthopoxvirus variola, Orthopoxvirus vaccinia, Orthopoxvirus simiae, Orthopoxvirus bovis, Orthopoxvirus muris, Orthopoxvirus cameli, Raccoonpox virus or Taterapox virus. Preferably, the viral late promoter element is one that is naturally found in virus of the species Orthopoxvirus vaccinia. According to a more preferred embodiment the viral late promoter element is one that is naturally found in a Modified Vaccinia Ankara Virus (MVA). Alternatively, the viral late promoter element is a structural variant of such a promoter element. Preferably such variants have at least a 60%, more preferably at least 70%, more preferably at least 80%, more preferably at least 90%, and even more preferably at least 95% nucleic acid sequence identity to a naturally occurring viral late promoter element and lead at least to the same level of transcription of a gene under the control of that variant viral late promoter element as the natural occurring viral late promoter element. Transcription levels of a gene under control of such a promoter can be determined using art known methods including in particular quantitative PCR (qPCR) of cDNA generated from the RNA isolated form a cell infected with the viral vector.

In a preferred embodiment of the recombinant Orthopox viral vector of the first aspect each of the viral early promoter elements is independently selected of the group consisting of:
(i) a Vaccinia Virus (VV) p7.5 early promoter element;
(ii) a viral early promoter element comprising or consisting of the nucleic acid sequence AAN¹N²AN³TGAAN⁴N⁵N⁶N⁷N⁸A (SEQ ID NO: 1), wherein each of N¹, N², N⁴, N⁵ and N⁶ is independently selected from A or T, preferably A, N³ is selected from C, G or T, preferably T, N⁷ is selected from C and A, preferably C and N⁸ is selected from A, C and T, preferably T, thus in a particularly preferred embodiment N¹, N², N⁴, N⁵ and N⁶ are A, N³ is T, N⁷ is C, and N⁸ is T;
   preferably the viral early promoter element comprises or consists of the nucleic acid sequence AAN¹N²AN³TGAAN⁴N⁵N⁶N⁷N⁸AN⁹TCTAATTTATTGN¹⁰AN¹¹GG (SEQ ID NO: 2), wherein each of N¹, N², N⁴, N⁵, and N⁶ is independently selected from A or T, preferably A, N³ is selected from C, G or T, preferably T, N⁷ is selected from C and A, preferably C, N⁸ is selected from A, C and T, preferably T, N⁹ is selected from G and T, preferably G, N¹⁰ is selected from C and T, preferably C, and N¹¹ is selected from A and C, preferably C, thus in a particularly preferred embodiment N¹, N², N⁴, N⁵, and N⁶ are A, N³ is T, N⁷ is C, N⁸ is T, N⁹ is G, N¹⁰ is C, and N¹¹ is C;
   preferably the viral early promoter element comprises or consists of the nucleic acid sequence AAN¹N²AN³TGAAN⁴N⁵N⁶N⁷N⁸AGTCTAATTTATTGCACGG (SEQ ID NO: 3), wherein each of N¹, N², N⁴, N⁵ and N⁶ is independently selected from A or T, preferably A, N³ is selected from C, G or T, preferably T, N⁷ is selected from C and A, preferably C, and N⁸ is selected from A, C and T, preferably T, thus in a particularly preferred embodiment N¹, N², N⁴, N⁵ and N⁶ are A, N³ is T, N⁷ is C, and N⁸ is T;
   preferably an early promoter element with the nucleic acids sequence according to any one of SEQ ID NO: 4 to 11 and most preferably SEQ ID NO: 4; and/or
(i) a Vaccinia Virus (VV) p7.5 late promoter element;
(ii) a late promoter element comprising or consisting of the nucleic acid sequence TTTN¹N²N³N⁴N⁵N⁶N⁷N⁸N⁹TTTTTN¹⁰N¹¹N¹²N¹³N¹⁴N¹⁵N¹⁶ATAAATA (SEQ ID NO: 41), wherein each of N¹ to N⁹ is independently selected from A, C, G, or T or absent, preferably T, and each of N¹⁰ to N¹⁶ is independently selected from A, C, G, or T or absent, preferably having the sequence GGCAT;
   preferably the viral late promoter element comprises or consists of the nucleic acid sequence TTTN¹N²N³N⁴N⁵N⁶N⁷N⁸N⁹TTTTTN¹⁰N¹¹N¹²N¹³N¹⁴N¹⁵N¹⁶ATAAATA (SEQ ID NO: 42), wherein N¹ is selected from C or T, preferably T, each of N² and N⁹ is independently selected from A, G or T, preferably T, each of N³, N⁴ and N⁸ is independently selected from A or T, preferably T, N⁵ is selected from G, T or absent, preferably T, N⁶ is selected from A, or T or absent, preferably T, N⁷ is selected from A, G, or T or absent, preferably T, N¹⁰ is selected from C, G, or T, preferably G, N¹¹ is selected from A, G or T, preferably G, N¹² is selected from A, or C, preferably C, N¹³ is selected from T or absent, preferably absent, N¹⁴ is selected from G or absent, preferably absent, N¹⁵ is selected from A, C, or T, preferably A, and N¹⁶ is selected from A, C, or T, preferably T, preferably a late promoter element with the nucleic acids sequence according to any one of SEQ ID NO: from 12 to 16, most preferably SEQ ID NO: 12;
      wherein each late and/or early promoter element is optionally connected by a nucleic acid linker, preferably of a length of 5, 6 or 7 nucleotides.

In a preferred embodiment all early promoter elements within the a first promoter of the Orthopox viral vector according to the first aspect of the invention are the same.

In a particularly preferred embodiment of the Orthopox viral vector according to the first aspect of the invention the first promoter comprises or consists of a nucleic acid according to SEQ ID NO: 18 to 30.

The first promoter may also comprise or consist from at least two early elements up to ten early elements. Preferably, the first promoter comprises or consists from at least three early elements up to seven early elements. More preferably, the first promoter comprises or consists from at least four early elements and up to 6 early elements. Most preferably, the first promoter comprises or consists of four early elements.

According to one embodiment, the at least one early promoter element is selected according to the generalized consensus SEQ ID NO: 1. According to a preferred embodiment, the first promoter comprises or consists of at least one early promoter element. According to a more preferred embodiment, the early promoter elements comprise or consist of any one of SEQ ID Nos: 4 to 11 and SEQ ID Nos: 43 to 46. According to an even more preferred embodiment, the early promoter element comprises or consists of any one of SEQ ID NO: 4, 5, 43, 44, 45 and 46. According to a most preferred embodiment, the early promoter element comprises or consists of SEQ ID NO: 4.

According to a preferred embodiment, the first promoter comprises or consists at least three early promoter elements, wherein the at least one early promoter element comprises the nucleotide motif AAN¹N²AN³TGAAN⁴N⁵N⁶N⁷N⁸A, wherein each of N¹, N², N⁴, N⁵ and N⁶ is independently selected from A or T (preferably A), N³ is selected from C, G or T (preferably T), N⁷ is selected from C and A (preferably C) and N⁸ is selected from A, C and T (preferably T), and at least one late promoter element, wherein the at least one late promoter element comprises the nucleotide motif TTTN¹N²N³N⁴N⁵N⁶N⁷N⁸N⁹TTTTTN¹⁰N¹¹N¹²N¹³N¹⁴N¹⁵N¹⁶ATAAATA, wherein each of N¹ to N⁹ is independently selected from A, C, G, T or absent (preferably T) and each of N¹⁰ to N¹⁶ is independently selected from A, C, G, T or absent (preferably having the sequence GGCAT).

According to an even more preferred embodiment, the first promoter comprises or consists of at least four early promoter elements, wherein each early promoter element comprises the nucleotide motif AAN¹N²AN³TGAAN⁴N⁵N⁶N⁷N⁸A, wherein each of N¹, N², ⁴, N⁵ and N⁶ is independently selected from A or T (preferably A), N³ is selected from C, G or T (preferably T), N⁷ is selected from C and A (preferably C) and N⁸ is selected from A, C and T (preferably T).

and at least one late promoter element wherein the at least one late promoter element comprises the nucleotide motif TTTN¹N²N³N⁴N⁵N⁶N⁷N⁸N⁹TTTTTN¹⁰N¹¹N¹²N¹³N¹⁴N¹⁵N¹⁶ATAAATA, wherein each of N¹ is selected from C or T (preferably T), N² and N⁹ is independently selected from A, G or T (preferably T), N³, N⁴ and N⁸ is independently selected from A or T (preferably T), N⁵ is selected from G, T or absent (preferably T), N⁶ is selected from A, T or absent (preferably T), N⁷ is selected from A, G, T or absent (preferably T), N¹⁰ is selected from C, G, T (preferably G), N¹¹ is selected from A, G or T (preferably G), N¹² is selected from A, or C (preferably C), N¹³ is selected from T or absent (preferably absent), N¹⁴ is selected from G or absent (preferably absent), N¹⁵ is selected from A, C, T (preferably A) and N¹⁶ is selected from A, C, T (preferably T). Preferably, the promoter elements in the first promoter operably linked to the first nucleic acid sequence encoding at least one immunostimulatory protein are 5′ prime of the first nucleic acid sequence encoding at least one immunostimulatory protein in the following order 5′-[late promoter element]-[early promoter element]ₙ-[first nucleic acid sequence encoding at least one immunostimulatory protein], wherein n is ≥4, preferably 4 to 6.

In this regard, the early promoter elements and the late promoter elements may be the same or different, respectively. It is preferred that all early promoter elements within the viral vector of the present invention are the same.

The first promoter may also comprise or consist of more than one late promoter elements. For example, the first promoter may comprise or consist from at least two promoter late elements up to five late promoter elements. Preferably, the first promoter comprises or consists of one late promoter element.

According to another embodiment, the individual late and early promoter elements are connected by a short linker. According to a preferred embodiment, the linker has a length of 2 to 12 nucleotides; more preferably, the linker has a length of 3 to 8 nucleotides: even more preferably, the linker has a length of 6 to 8 nucleotides. According to a most preferred embodiment, the linker has a length of 5 to 8 nucleotides. Preferably, the linker has a sequence selected from TCCGGT, TCCGGA, TCTGGA, TCTCGT, ATAGGA or AGCTT. The linkers connecting individual late and early promoter elements in a promoter do not have to be the same.

According to a preferred embodiment, the first promoter comprises or consists of a sequence according to any one of SEQ ID Nos: 18 to 30. According to a more preferred embodiment, the first promoter comprises or consists of a sequence according to any one of SEQ ID Nos: 18, 19, 22, 23 or 24. According to an even more preferred embodiment, the first promoter comprises or consists of a sequence according to any one of SEQ ID Nos: 18, 19 or 23. According to the most preferred embodiment, the first promoter comprises or consists of the sequence according to SEQ ID NO: 18.

In a second aspect, the invention provides a cell comprising the recombinant Orthopox viral vector according to the first aspect of the invention. Typically, such a cell is transformed, transduced or transfected with a viral vector of the first aspect of the invention. Cells that receive and subsequently express foreign nucleic acids or vectors consisting of DNA or RNA by the process of transformation or transduction have been “transformed” or “transduced”. Preferably, the cell of the invention comprises a viral vector as described above. The cell can be a eukaryotic cell, e.g., mammalian cell (e.g. a human cell), yeast, plant, animal, fungi, or algae, or can be a prokaryotic cell, e.g., bacteria or protozoa. Preferably, the cell is a mammalian cell such as a lymphocyte or leukocyte, more preferably a macrophage. Preferably, the cells carry a scavenger receptor on their surface. More preferably, the scavenger receptor is a scavenger receptor of type 1. Most preferably, the scavenger receptor is MARCO.

According to a third aspect, the invention provides a composition, comprising
a) the recombinant Orthopox viral vector according to the first aspect of the invention, or a cell comprising the viral vector of the first aspect of the invention; and
b) a plurality of two or more Orthopox viral vectors encoding different therapeutic proteins according to the first aspect of the invention; and
c) a pharmaceutically acceptable carrier; and optionally
d) a recombinant viral vector comprising a nucleic acid sequence encoding a checkpoint inhibitor, a nucleic acid encoding a checkpoint inhibitor or a checkpoint inhibitor.

In one embodiment the composition comprises a therapeutically effective amount of recombinant Orthopox viral vector according to the first aspect of the invention or the cell according to the second aspect of the invention, a therapeutically effective amount of a a plurality of two or more Orthopox viral vectors encoding different therapeutic proteins according to the first aspect of the invention, together with a suitable amount of a pharmaceutically acceptable carrier and/or excipient so as to provide the form for proper administration to a subject. The formulation of the composition should suit the mode of administration. For example, for intravenous administration, it is preferred that the carrier is an aqueous carrier. According to an alternative embodiment, the composition may optionally comprise further recombinant viral vectors comprising a nucleic acid sequence encoding a checkpoint inhibitor, a nucleic acid encoding a checkpoint inhibitor or a checkpoint inhibitor. Preferably, the checkpoint inhibitor is selected from the group consisting of CTLA-4 inhibitors, PD-1 inhibitors, and PD-L1 inhibitors. More preferably, the checkpoint inhibitor is a PD-1 inhibitor. Even more preferably, the PD-1 inhibitor is an anti-PD1 antibody selected from the group consisting of Nivolumab, Atezolizumab, Pembrolizumab, Cemiplimab, Durvalumab and Avelumab.

According to one embodiment, the pharmaceutically acceptable carrier is an aqueous carrier. Preferably, the aqueous carrier is selected from the group consisting of sterile liquids, such as saline solutions in water and oils, including but not limited to those of petroleum, animal, vegetable or synthetic origin, such as peanut oil, soybean oil, mineral oil, sesame oil and the like.

In one embodiment the pharmaceutical composition may further comprise a therapeutic agent or pharmacologically active substance such as but not limited to adjuvants and/or additional active ingredients, in a pharmaceutically or physiologically acceptable formulation selected to be suitably administered according to the selected mode of administration. Non-limiting examples of suitable adjuvants alum, aluminum phosphate, aluminum salts, aluminum hydroxide, aluminum silica, calcium phosphate, incomplete Freund's adjuvant, QS21, MPL-A, RIBI DETOX™, and/or combinations thereof.

In one embodiment the pharmaceutical compositions can take the form of solutions, suspensions, emulsion, tablets, pills, capsules, powders, sustained-release formulations and the like. For preparing pharmaceutical compositions of the present invention, pharmaceutically acceptable carriers can be either solid or liquid and are preferably liquid. Liquid form compositions include solutions, suspensions, and emulsions, for example, water, saline solutions, aqueous dextrose, glycerol solutions or water/propylene glycol solutions. For parenteral injections (e.g. intravenous, intraarterial, intraosseous infusion, intramuscular, subcutaneous, intraperitoneal, intradermal, and intrathecal injections), liquid preparations can be formulated in solution in, e.g. aqueous polyethylene glycol solution. A saline solution is a preferred carrier when the pharmaceutical composition is administered intravenously.

In one embodiment, the composition is in a unit dosage form. In such form the composition may be subdivided into unit doses multi doses containing appropriate quantities of the active component. The unit dosage form can be a packaged composition, the package containing discrete quantities of the composition, such as packaged tablets, capsules, and powders in sealed vials or ampoules. Also, the unit dosage form can be a capsule, an injection vial, a tablet, a cachet, or a lozenge itself, or it can be the appropriate number of any of these in packaged form. The composition, if desired, can also contain minor amounts of wetting or emulsifying agents, or pH buffering agents. The compositions may be stored in a freeze-dried (lyophilized) condition requiring only the addition of a sterile liquid carrier, for example water for injections, immediately prior to use.

The form of the compositions, the route of administration, the dosage and the regimen naturally depend upon the condition to be treated, the severity of the illness, the age, weight, and gender of the subject, the desired duration of the treatment etc. The compositions of the present invention may be in any suitable form, depending upon the desired method of administering it to a subject.

In one embodiment the pharmaceutical compositions of the fourth aspect of the invention contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

To prepare compositions of the present invention, an effective amount of the recombinant Orthopox viral vector according to the first aspect of the invention or of the cell according to the second aspect of the invention, and an effective amount of a plurality of two or more Orthopox viral vectors according to the first aspect of the present invention may be dispersed in a pharmaceutically acceptable carrier or aqueous medium. Optionally, a further recombinant viral vector comprising a nucleic acid sequence encoding a checkpoint inhibitor, a nucleic acid encoding a checkpoint inhibitor or a checkpoint inhibitor can be added.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that it is easy to syringe. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. Sterile injectable solutions are prepared by incorporating the active compounds in the required amount in the appropriate solvent with various of the other ingredients enumerated above, as required. followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention provides in fourth aspect, a recombinant Orthopox viral vector, a cell comprising the recombinant Orthopox viral vector according to the first aspect of the invention, or a composition according to the third aspect of the invention for use in medicine.

The invention provides in a fifth aspect, a recombinant Orthopox viral vector according to the first aspect of the invention, a cell according to the second aspect of the invention, or a composition according to the third aspect of the invention for use in treating, ameliorating or preventing cancer.

According to one embodiment, the cancer is a breast cancer, small intestine cancer, stomach cancer, kidney cancer, bladder cancer, uterus cancer, ovarian cancer, testes cancer, lung cancer, colon cancer, prostate cancer, a B cell lymphoma, a Burkitt's lymphoma or a Hodgkin's lymphoma.

Further non-limiting examples of cancers envisaged in the present invention include: adrenocortical carcinoma, AIDS-related cancers, AIDS-related lymphoma, anal cancer, appendix cancer, astrocytomas, neuroblastoma, basal cell carcinoma, bile duct cancer, bone cancers, brain tumours, such as cerebellar astrocytoma, cerebral astrocytoma/malignant glioma, ependymoma, medulloblastoma, supratentorial primitive neuroectodermal tumours, visual pathway and hypothalamic glioma, bronchial adenomas, central nervous system lymphoma, cerebellar astrocytoma, cervical cancer, cutaneous T-cell lymphoma, desmoplastic small round cell tumour, endometrial cancer, ependymoma, oesophageal cancer, Ewing's sarcoma, germ cell tumours, gallbladder cancer, gastric cancer, gastrointestinal carcinoid tumour, gastrointestinal stromal tumour, gliomas, head and neck cancer, heart cancer, hepatocellular (liver) cancer, Hodgkin lymphoma, Hypopharyngeal cancer, intraocular melanoma, islet cell carcinoma, Kaposi sarcoma, laryngeal cancer, lip and oral cavity cancer, liposarcoma, liver cancer, non-small cell and small cell lung cancer, lymphomas, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumour, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, rectal cancer, renal cell carcinoma, renal pelvis and ureter transitional cell cancer, retinoblastoma, rhabdomyosarcoma, salivary gland cancer, sarcomas, skin cancers, merkel cell carcinoma, soft tissue sarcoma, squamous cell carcinoma, T-cell lymphoma, throat cancer, thymoma, thymic carcinoma, thyroid cancer, trophoblastic tumour (gestational), cancers of unknown primary site, urethral cancer, uterine sarcoma, vaginal cancer, vulvar cancer, Waldenström's macroglobulinemia, and Wilms tumour (nephroblastoma).

In preferred embodiments, the cancer comprises a solid tumour. In this regard, while lymphomas are not generally considered solid tumours, accessible solid tumour forms of lymphomas may form in the lymph node.

The recombinant Orthopox viral vector or the composition as described herein may be administered in a therapeutically effective amount to a subject in need of treating cancer. Typically, the recombinant Orthopox viral vectors of the present invention will be packaged into viral particles and the particles will then be delivered to the site of the tumour. For example, when the recombinant viral vector is administered as an active ingredient of a pharmaceutical composition, the dosage of recombinant virus or viral particle is represented in plaque forming units (PFU) of the virus or viral particle. The term “plaque forming unit” refers to the amount of virus particle capable of forming plaques per unit volume. Suitable amounts may be, for example, 10² to 10¹⁴ PFU, preferably 10⁵ to 10¹² PFU, and more preferably 10⁶ to 10¹⁰ PFU. The recombinant Orthopox viral vector or the composition as described herein may be administered more than once. One cycle of treatment can correspond to twice, three times, or four times administration every day or at weekly or biweekly or monthly intervals. Treatment cycles can be repeated several times to obtain complete cure.

It is to be understood that the amounts of the recombinant Orthopox viral vector according to the first aspect of the invention, the cell according to the second aspect of the invention, or the composition according to the third and fourth aspects of the present invention may vary depending on the specific compound being used, the particular compositions formulated, the mode of application, the size and type of the tumour, and the recipient of the compound. The recombinant Orthopox viral vector according to the first aspect of the invention, the cell according to the second aspect of the invention, or the composition according to the third and fourth aspects of the present invention vector may be administered only once or repeatedly.

One suitable route of administration is by injection of the viral particles in a sterile solution. The particles may be administered alone. It is preferable to present the viral particles as pharmaceutical composition or formulation. Thus, the composition preferably comprises a viral particle, together with one or more acceptable carriers and optionally other therapeutic ingredients as mentioned above. The carrier must be “acceptable” in the sense of being compatible with the other ingredients of the composition or formulation and not deleterious to the recipients thereof.

According to one embodiment, the recombinant Orthopox viral vector according to the first aspect of the invention, the cell according to the second aspect of the invention, or the composition according to the third and fourth aspects of the present invention may be administered directly into the tumour tissue. According to a preferred embodiment, the recombinant Orthopox viral vector according to the first aspect of the invention, the cell according to the second aspect of the invention, or the composition according to the third and fourth aspects of the present invention are administered by direct injection, or with a catheter prior or during surgery. The recombinant Orthopox viral vector according to the first aspect of the invention, the cell according to the second aspect of the invention, or the composition according to the third and fourth aspects of the present invention may also be administered by regional perfusion, direct intratumoural direction, direct direction into a body cavity (intracaviterial administration), for example by intra-peritoneum injection. Preferably, the routes of administration are parenteral injection, e.g. intradermal injection or intramuscular injection.

Further routes of administration include, but are not limited to topical, oral, enteral, nasal (i.e. intranasal), inhalation, intrathecal, rectal, vaginal, intraocular, subconjunctival, sublingual, intradermal, transdermal, or parenteral administration, including subcutaneous, percutaneous, intravenous, intramuscular, intratumoural, intranodal, intrasternal, intracavernous, intravesical, or intraurethral injection or infusion.

According to one embodiment, the recombinant Orthopox viral vector and the recombinant viral vector comprising a nucleic acid sequence encoding a checkpoint inhibitor, a nucleic acid encoding a check-point inhibitor or a checkpoint inhibitor are administered simultaneously or subsequently. For example, the recombinant Orthopox viral vector and the recombinant viral vector comprising a nucleic acid sequence encoding a checkpoint inhibitor, a nucleic acid encoding a checkpoint inhibitor or a checkpoint inhibitor may be administered sequentially, e.g. at hours, daily, weekly or monthly intervals, or in response to a specific need of the subject. In certain embodiments, the recombinant Orthopox viral vector is administered at least one, at least two, at least three, at least four, at least five, at least six, least seven, at least eight, at least nine, at least ten, or at least 12 hours, or at least 18 hours prior to administration of the recombinant viral vector comprising a nucleic acid sequence encoding a checkpoint inhibitor, a nucleic acid encoding a checkpoint inhibitor or a checkpoint inhibitor. In other particular embodiments, the recombinant Orthopox viral vector is administered at least one, at least two, at least three, at least four, at least five, at least six, or at least seven days prior to administration of the recombinant viral vector comprising a nucleic acid sequence encoding a checkpoint inhibitor, a nucleic acid encoding a checkpoint inhibitor or a checkpoint inhibitor. In other embodiments, the recombinant Orthopox viral vector is administered between one and 36 days prior to administration of the recombinant viral vector comprising a nucleic acid sequence encoding a checkpoint inhibitor, a nucleic acid encoding a checkpoint inhibitor or a checkpoint inhibitor. The recombinant Orthopox viral vector may be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 days prior to administration of the recombinant viral vector comprising a nucleic acid sequence encoding a checkpoint inhibitor, a nucleic acid encoding a checkpoint inhibitor or a checkpoint inhibitor. In still other embodiments, the recombinant Orthopox viral vector and the recombinant viral vector comprising a nucleic acid sequence encoding a checkpoint inhibitor, a nucleic acid encoding a checkpoint inhibitor or a checkpoint inhibitor are each administered sequentially to the subject in need thereof, more than once (e.g. twice, three times, or four times).

### FIGURES

FIG. 1. Efficacy of MVA-SynE1-IL12 and MVA-7.5-IL12 injected intratumourally (it) in anti-PD1 resistant tumours. Tumour bearing mice (LLC tumours) were treated it with MVA encoding IL12 under the control of a synthetic early promoter (MVA-SynE1-IL12) or MVA-IL12 under the control of the 7.5 promoter (MVA-7.5-IL12) at the dose of 10e7 infectious units (ifu) in combination to anti-PD1 antibody injected intraperitoneally (ip). Treatments started at day 0, on mice randomized according to tumour volume. Injection of MVA was repeated ad day 2 and 4, while the anti-PD1 treatment was performed twice per week until day 17. Tumour growth over time is shown for individual mice belonging to the different groups of treatment. Dashed lines represent non-responder mice.

FIG. 2. Efficacy of MVA-SynE1-IL12 at low dose in presence anti-PD1 treatment. Tumour bearing mice (LLC tumours) were treated with MVA encoding IL12 under the control of a synthetic early promoter (MVA-SynE1-IL12) at the dose of 10e7 infectious units (ifu) or 2×10e5 ifu, in presence of anti-PD1 antibody injected intraperitoneally (ip). Treatments started at day 0, on mice randomized according to tumour volume. Injection of MVA was repeated ad day 2 and 4, while the anti-PD1 treatment was performed twice per week until day 17. Shown are the curves of tumour growth over time.

FIG. 3. Efficacy of MVA-SynE1-IL12 as stand-alone treatment at low dose treatment. Tumour bearing mice (LLC tumours) were treated with MVA encoding IL12 under the control of a synthetic early promoter (MVA-SynE1-IL12) at the dose of 10e7 infectious units (ifu) or 2×10e5 ifu (monotherapy treatments). Treatments started at day 0, on mice randomized according to tumour volume. Injection of MVA was repeated ad day 2 and 4, while the anti-PD1 treatment was performed twice per week until day 17. Shown are the curves of tumour growth over time.

FIG. 4. Efficacy of MVA-SynE1-IL12 versus Ad-IL12 in a model of peritoneal carcinomatosis. CT26 tumour cells were injected ip in BA1BC mice 3 days before the start of treatment. At day 0, mice were treated with MVA encoding IL12 under a synthetic early promoter (MVA-SynE1-IL12) at the dose of 10e7 infectious units (ifu) or with an equivalent dose of Adeno encoding IL12. Treatment was repeated at day 2 and 4. Overall survival is shown for control mice (untreated), MVA-SynE1-IL12 and Ad-IL12 treated mice.

FIG. 5. In vitro expression of IL12 in Hela cells infected with Adeno or MVA encoding IL12. HeLa cells were infected with the indicated vectors encoding IL12 at 1 MOI (1 infectious units/cell). Supernatant was collected 24 h post infection and subjected to IL12 Elisa assay. For MVA infected cells results are the average SD of two independent experiments.

FIG. 6. Intratumoural expression of IL12 upon in vivo treatment in mice. Tumour bearing mice (LLC tumours) were treated it with a single injection of Adeno encoding IL12 (Ad-IL12), MVA encoding IL12 under the natural 7.5 promoter (MVA-p7.5-IL12), or MVA encoding IL12 under a synthetic early promoter (MVA-SynE1-IL12) at the dose of 10⁷ ifu. Expression of IL12 from harvested tumours was measured overtime by ELISA assay and expressed as pg/ug protein lysates.

FIG. 7. Expression of IL12 in muscle versus tumour upon in vivo treatment. Mice were injected with MVA encoding IL12 under a synthetic early promoter (MVA-SynE1-IL12) at the dose of 10⁷ ifu, given intramuscular or intratumoural. Expression of IL12 from tumours versus muscle was measured by ELISA assay and expressed as pg/ug protein lysates.

FIG. 8. Levels of intratumoural M1 and M2 macrophages upon intratumoural treatment with MVA-IL12. Tumour bearing mice (LLC tumours) were injected intratumourally with MVA encoding IL12 under a synthetic early promoter (MVA-SynE1-IL12) or MVA mock at the dose of 10⁷ ifu, given at day 0, day 2, and day 4. Tumours were harvested at day 7 and analysed by flow cytometry to measure the levels of M1 and M2 macrophages following treatments.

FIG. 9. Schematic representation of the arrangements of P7.5, SynE1 and SynE2 promoters. 7.5 L: late element from P7.5 promoter; 7.5E: early element from P7.5 promoter; sL: synthetic late element (SEQ ID NO: 12); 7.5E^{mod}, modified early element derived from P7.5 promoter (SEQ ID NO: 4). Arrows indicate direction of transcription.

The invention is described by way of the following examples which are to be construed as merely illustrative and not limitative of the scope of the invention.

FIG. 10. Efficacy of MVA-SynE1-IL12 alone and in combination with anti-PD1 in B16F10 tumor model resistant to anti-PD1 activity. Survival curves of C57BL/6 mice s.c. injected with B16F10 cells. Mice (n=9 per group) with established tumors were randomized and treated it with MVA encoding IL12 under the control of a synthetic early promoter (MVA-SynE1-IL12) (dose of 6×10⁵ IFU, 2 cycles of 4 injections, every 4 days starting from day 0) alone or in combination with anti-PD1 antibody injected intraperitoneally twice per week until day 17. Anti-PD1 treated mice were used as control group.

FIG. 11. MVA-SynE1-IL12 IT injection controls tumor growth of distant un-injected tumors, (a-f) C57BL/6 mice were challenged s.c. with MC38 cells on both flanks (bilateral tumor implantation). Mice with established tumors were randomized according to similar tumor volumes (n=7-10 mice per group). One tumor (right flank) was injected i.t with MVA-mIL12 at 10⁷ IFU (a) or MVA-mIL12 at 6×10⁵ ifu (b) (2 cycles of 4 injections, every 4 days starting from day 0) in combination with anti-PD1, the other one (left flank) didn't receive any IT treatment (d, e). Mice treated with anti-PD1 only were used as control for both flanks. Tumor volume was monitored over time. Lines in the graphs represent each individual tumor (full lines, responder tumors; dot lines, non-responder tumors). Percentages on the graphs indicate the response rate (complete response, CR).

FIG. 12. SynE promoters comprising consensus SEQ ID NO: 1 or SEQ ID NO: 3 drive stronger early transgene expression compared to P7.5, (a-b) Schematic representation of the different SynE promoters tested. The identity of the early (black box) and late (white box) promoter elements is indicated in each box, with their respective SEQ ID NOs where applicable. The consensus SEQ ID NO:3 comprises early elements SEQ ID NO: 4 and SEQ ID NO: 5. The nucleotide sequence of early elements Early-A (SEQ ID NO:43), Early-B (SEQ ID NO:44), Early-C (SEQ ID NO:45) and Early-D (SEQ ID NO:46) is indicated in b) and include a sequence comprised by the more generalized consensus SEQ ID NO: 1, with fixed nucleotide positions in SEQ ID NO: 1 indicated in bold. 7.5 L: late element from P7.5 promoter; 7.5E: early element from P7.5 promoter; SynE1 corresponds to SEQ ID NO: 18; SynE2 corresponds to SEQ ID NO: 19; SynE5 corresponds to SEQ ID NO: 22; SynE7 corresponds to SEQ ID NO: 24. Arrows indicate direction of transcription. (c) To test early promoter activity, Hela cells were infected with MVA-Red (an MVA vector encoding the reporter HcRed protein, whose expression was not assessed in this experiment) and transfected, 2.5 h later, with plasmids encoding mIL12 under the control of the different SynE promoters or the P7.5 promoter. AraC (40 ug/ml) was added at the time of infection with MVA vectors to block intermediate/late expression. mIL12 levels were measured by ELISA on cell culture supernatant 4 h post transfection, and are expressed as fold of change relative to the levels measured in cells transfected with P7.5-mIL12. N=3 for P7.5, SynE1, SynE2 and SynE7; n=2 for SynE5, SynE14, SynE15, and SynE16. FOC=Fold-change.

FIG. 13. Description of SEQ ID NO: 1 in the WIPO ST.25 format.

### EXAMPLES

### Preparation of Recombinant Orthopox Viral Vectors

Methods are known to the person skilled in the art how an expression cassette or a promoter according to the present invention can be inserted into a viral genome, in particular into the genome of a vaccinia virus, most preferably into the genome of MVA. By way of example, the expression cassette or the promoter or derivative thereof according to the present invention may be inserted into the genome of MVA by homologous recombination. To this end a nucleic acid is transfected into a permissive cell line such primary bird cell lines or bird-derived cell lines, wherein the nucleic acid comprises the expression cassette or the promoter or derivative thereof according to the present invention flanked by nucleotide stretches that are homologous to the region of the MVA genome in which the expression cassette or the promoter or derivative thereof according to the present invention is to be inserted. The cells are infected by MVA and in the infected cells homologous recombination occurs between the nucleic acid and the viral genome. Alternatively, it is also possible to first infect the cells with MVA and then to transfect the nucleic acid into the infected cells. The recombinant MVA is then selected by methods known in the prior art. The construction of recombinant MVA is not restricted to this particular method. Instead, any suitable method known to the person skilled in the art may be used to this end.

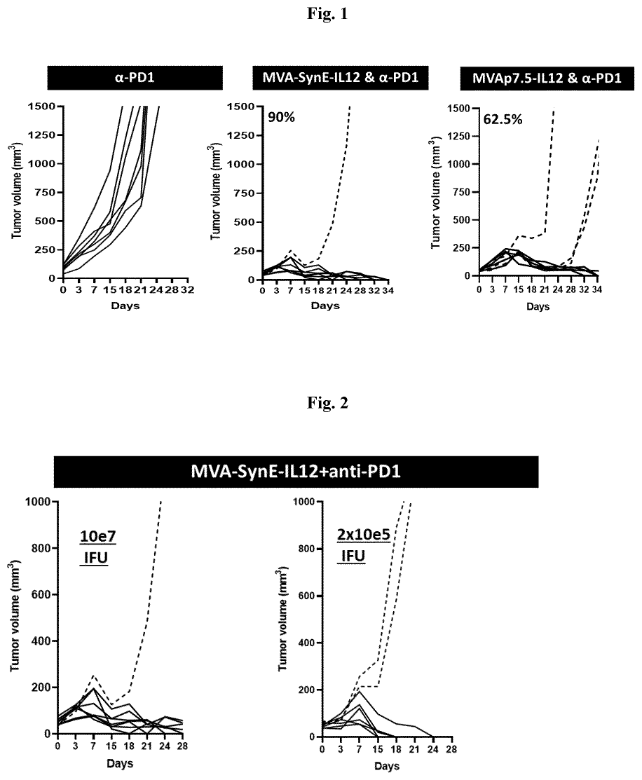

Example 1: Intratumoural treatment using MVA-IL12 combined with anti-PD1 treatment is highly effective in tumours resistant to checkpoint inhibitors (CPI), with MVA encoding single-chain murine IL-12 (sc-mIL12) (according to SEQ ID NO: 37) under the control of a synthetic promoter (SynE-1) (according to SEQ ID NO: 18) exerting a stronger effect as compared to the natural p7.5 promoter (SEQ ID NO: 17) (FIG. 1).

Tumour bearing mice (Lewis Lung Carcinoma model, LLC) were treated intratumourally (it) with MVA encoding IL12 under the control of a synthetic early promoter (MVA-SynE1-IL12) or MVA-IL12 under the control of the 7.5 promoter according to (MVA-7.5-IL12) at the dose of 10e7 infectious units (ifu) in combination to anti-PD1 antibody injected intraperitoneally (ip). Treatments started at day 0, on mice randomized according to tumour volume. Treatment with MVA was performed at fay 0, day 2 and 4, while the anti-PD1 treatment was performed twice per week until day 17. Tumour growth was measured over time using a digital caliper every 3-4 days. Tumour volume was calculated using the formula: 0.5×length×width², where the length was the longer dimension. Results show a very strong therapeutic effect of both MVA encoding IL-12 combined to anti-PD1, with tumour regressions and cure observed in 90% and 62.5% of mice treated with MVA-SynE1-IL12 and MVA-p7.5-IL12, respectively (FIG. 1) high-lighting stronger activity of MVA-SynE1-IL12. In this model, anti-PD1 treatment alone was not effective.

Example 2: Efficacy of intratumoural MVA-SynE1-IL12 is maintained even at low dose (FIG. 2).

Tumour bearing mice (Lewis Lung Carcinoma model, LLC) were treated intratumourally (it) with MVA encoding IL12 under the control of the SynE-1 synthetic early promoter (MVA-SynE1-IL12) at the dose of 10e7 infectious units (ifu) or 2×10e5 ifu, in presence of anti-PD1 antibody injected intraperitoneally (ip). For both groups of mice, treatments started at day 0, on mice randomized according to tumour volume. Injection of MVA was repeated at day 2 and 4, while the anti-PD1 treatment was performed twice per week until day 17. Results show an effective and potent anti-tumour effect also when lowering the dose of MVA-SynE1-IL12.

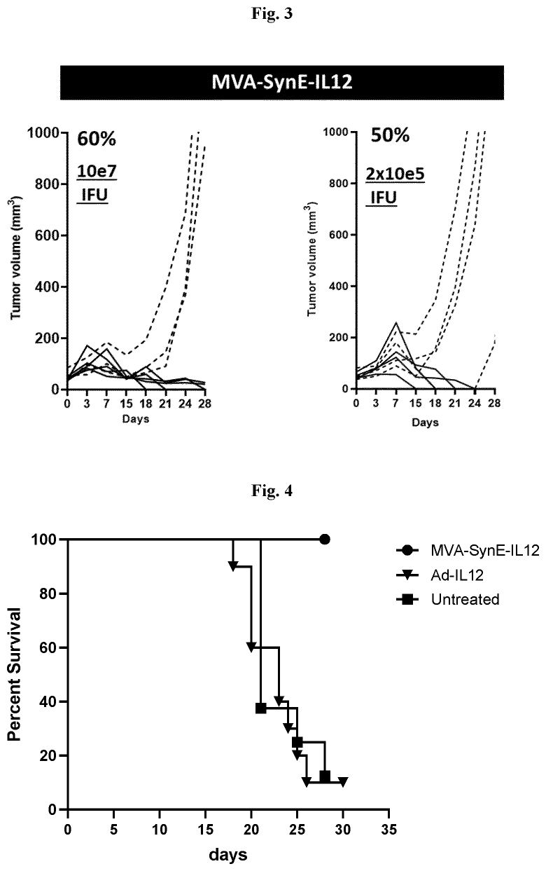

Example 3: MVA-SynE1-IL12 is active as stand-alone treatment (FIG. 3).

The anti-tumour effect of MVA-SynE1-IL12 as monotherapy was investigated in tumour bearing mice (same tumour model and treatment modality as reported in the Example 1 and 2), at the dose of 10e7 infectious units (ifu) or 2×10e5 ifu. Tumour volumes (mm3) were measured over time, demonstrating activity of MVA-SynE1-IL12 at both doses also in absence of anti-PD1 treatment.

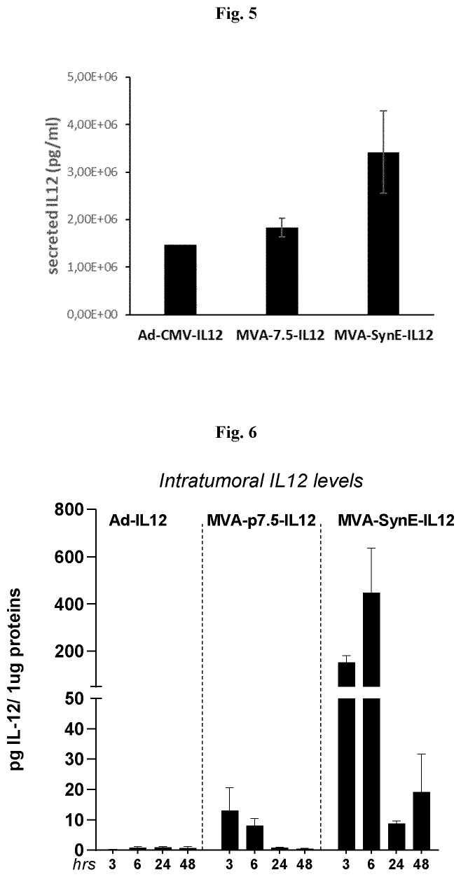

Example 4: Adenovirus vector encoding IL12 is not effective in a model of peritoneal carcinomatosis (FIG. 4).

The activity of MVA-SynE1-IL12 was compared to the one of a different viral vector, more specifically to Ad5-encoding sc-mIL-12 under the control of CMV promoter. CT26 (murine colon cancer cells) were injected in the peritoneum of BalBC mice. Three days later (day 0), mice were treated with MVA encoding IL12 under a synthetic early promoter (MVA-SynE1-IL12) at the dose of 10e7 infectious units (ifu) or with an equivalent dose of Ad5 encoding IL12. Treatment was repeated at day 2 and 4. Overall survival was monitored over time, compared to the survival of control untreated mice, showing efficient inhibition of peritoneal carcinomatosis by MVA-SynE1-IL12, but not by Ad-IL12 with a survival rate of 100% versus 10% at day 30, respectively.

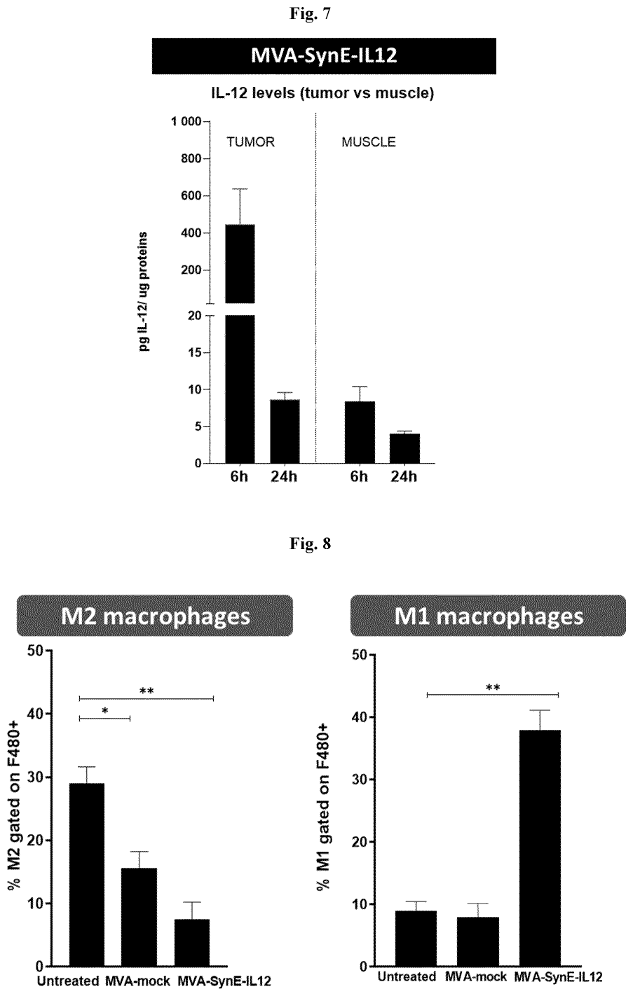

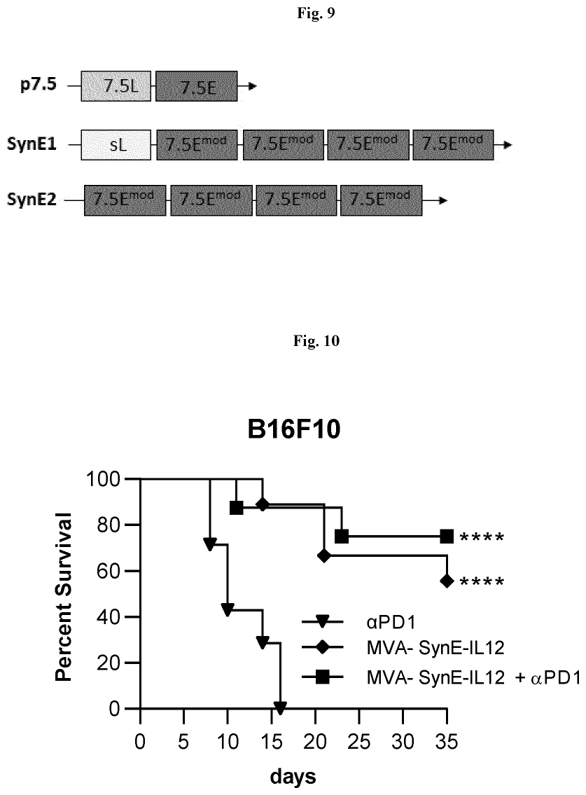

Example 5: Adenovirus vector and MVA vector encoding IL12 express similar cargo levels in vitro (FIG. 5).

The expression of IL12 produced by Adeno or MVA encoding IL12 was measured in vitro. HeLa cells were infected with MVA-SynE1-IL12 or MVA-p7.5-IL12, or Ad5-IL12 at 1 MOI (1 infectious units/cell). Supernatant was collected 24 h post infection and subjected to IL12 Elisa assay, showing similar levels of expression for all the 3 vectors.

Example 6: MVA-SynEl promoter drives very high IL12 expression when injected into the tumour (FIG. 6).

The levels of intratumoural IL12 produced by Adeno or MVA encoding IL12 were measured upon in vivo treatment in mice. Tumour bearing mice (LLC tumours) were treated with a single injection of Adeno encoding IL12 (Ad-IL12), MVA encoding IL12 under the natural 7.5 promoter (MVA-p7.5-IL12), or MVA encoding IL12 under a synthetic early promoter (MVA-SynE1-IL12) injected intratumourally at the dose of 10\hat{ }7 ifu. Expression of IL12 from harvested tumours was measured overtime by ELISA assay, showing that MVA-SynE1 promoter drives a very high IL12 expression with about 50-fold higher expression than MVA-p7.5 promoter. Levels of IL12 from Ad-IL12 treated tumours were very low (FIG. 6).

Example 7: MVA-SynEl promoter drives very high IL12 expression in the tumour but not in the muscle (FIG. 7).

Expression of IL12 was measured in muscle versus tumour upon in vivo treatment with MVA-SynE1-IL12. Mice were injected with a single injection of MVA-SynE1-IL12 at the dose of 10\hat{ }7 ifu, given intramuscular or intratumoural. Levels of IL12 from tumours versus muscle were measured by ELISA assay, demonstrating that MVA-SynEl promoter drives very high IL12 expression in the tumour but not in to the muscle (FIG. 7).

Example 8: Intratumoural treatment with MVA-SynE1-IL12 efficiently reprograms the tumour microenvironment by decreasing the levels of suppressive M2 macrophages, while increasing the amount of M1 pro-inflammatory macrophages (FIG. 8).

In this example, the frequency of M1 and M2 macrophages upon in vivo treatment with MVA-SynE1-IL12 or MVA mock vector was measured. Mice were injected with three consecutive injections of MVA-SynE1-IL12 or MVA-mock at the dose of 10\hat{ }7 ifu, given at day 0, 2 and 4. At day 7, tumours were harvested and the levels of M1 and M2 macrophages were assessed by flow cytometry assay, demonstrating that MVA-mock is able to reduce the M2 immunosuppressive cells, but not the M1 pro-inflammatory cells. Concomitant decrease of M2 with increase of M1 was achieved only upon treatment with MVA-SynE1-IL12 (FIG. 8).

Example 9: Intratumoral (IT) treatment with MVA-SynE1-IL12 alone and combined with anti-PD1 therapy is highly effective in B16F10 tumors resistant to checkpoint inhibitors (CPI).

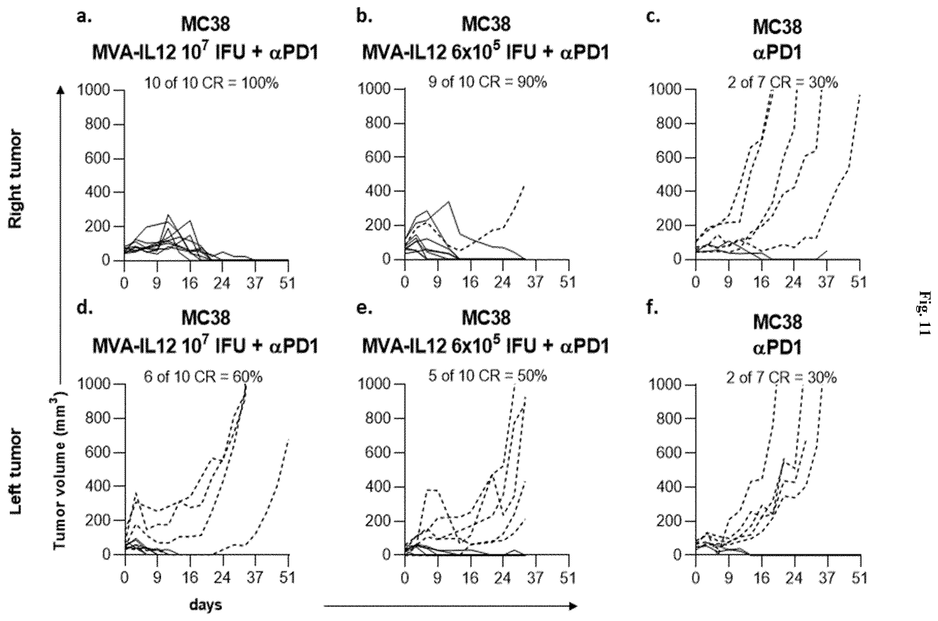

In this example, we investigated the therapeutic efficacy of MVA-SynE1-IL12 in the B16F10 model, a murine tumor model known to be resistant to the activity of CPI. Tumor bearing mice (n=9 per group) were treated intratumorally (it) with MVA encoding IL12 under the control of the SynE1 synthetic early promoter (MVA-SynE1-IL12) at the dose of 6×10e5 ifu, in presence of anti-PD1 antibody injected intraperitoneally (ip). For both groups of mice, treatments started at day 0, on mice randomized according to tumor volume. Injection of MVA was repeated every 4 days for a total of 8 injections, while the anti-PD1 treatment was performed twice per week until day 17. Results show an effective control of tumor growth upon treatment with MVA-SynE1-IL12, alone and combined with anti-PD-1, as compared to the control group receiving anti-PD1 monotherapy (FIG. 10).

Example 10: MVA-SynE1-IL12 IT immunotherapy controls tumor growth of both injected and distant, un-injected tumors.

To test the abscopal antitumor response induced by MVA-SynE1-IL12, a bilateral MC38 tumor implantation model was used to evaluate whether MVA-SynE1-IL12 has antitumor activity against distant, not injected tumors. Intratumoral injections of MVA-SynE1-IL12 at a dose of 6×10⁵ ifu or 10⁷ ifu were performed in one of the two masses of tumor bearing mice, in presence of anti-PD1 given ip. Combination of anti-PD1 and MVA-SynE1-IL12 resulted at both doses in complete tumor regression of most injected tumors (FIG. 11a-c). Distant, not injected tumors were also eradicated upon treatment in 50 and 60% of animals, at the dose of 6×10⁵ ifu and 10⁷ ifu, respectively, underlining the capability of MVA-SynE1-IL12 to control cancer growth also at distant sites (FIG. 11d-f).

Example 11: Strong early in vitro activity of SynE promoters comprising consensus SEQ ID NO:1 or SEQ ID NO: 3.

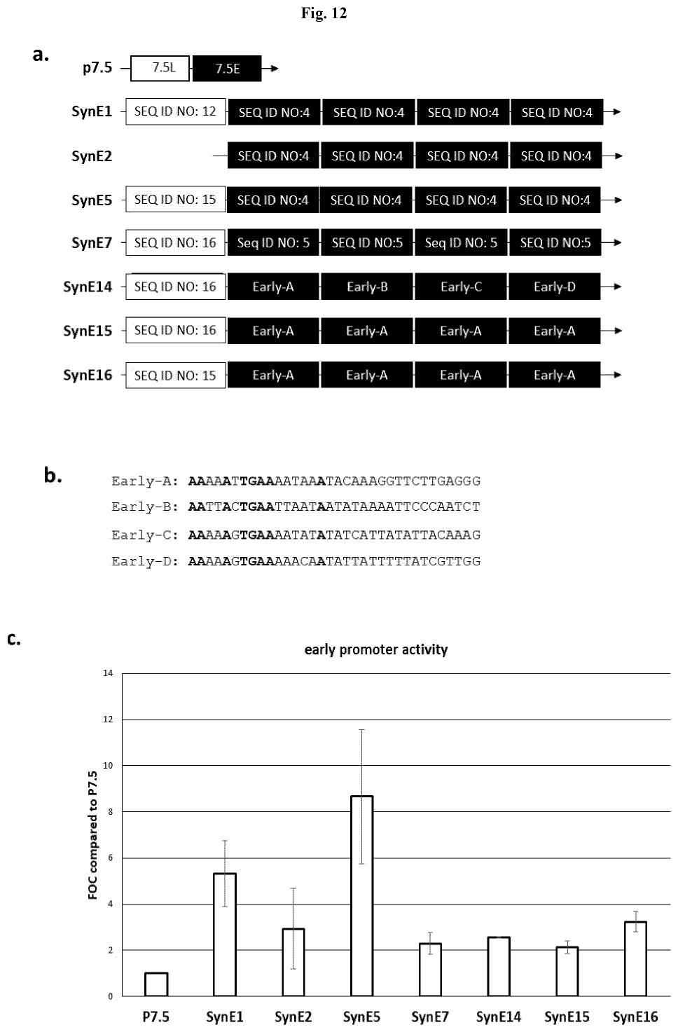

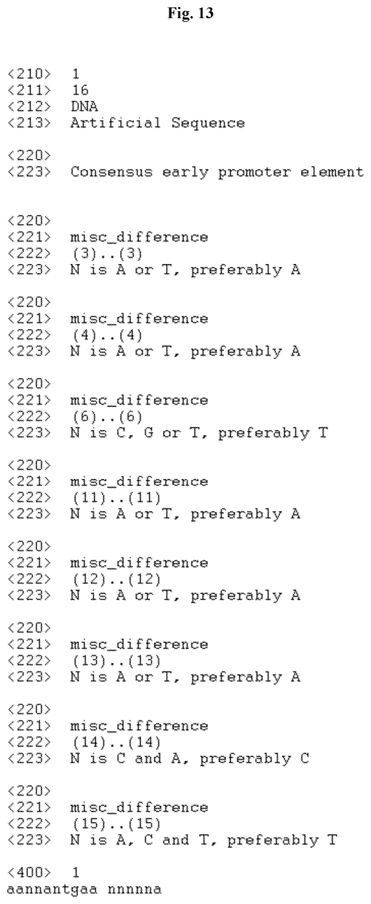

This example compares the early activity of the P7.5 promoter with SynE promoters containing different early elements, consisting of consensus SEQ ID NO:3 (early elements SEQ ID NO: 4 and SEQ ID NO: 5) or comprising the more generalized consensus SEQ ID NO: 1: early elements Early-A (SEQ ID NO: 43), Early-B (SEQ ID NO: 44), Early-C (SEQ ID NO: 45) or Early-D (SEQ ID NO: 46), combined or not with different late elements (FIGS. 12a and b).

To this aim HeLa cells were infected with MVA to deliver all viral functions needed for poxviral promoter expression and transfected with plasmids encoding mIL12 under the control of the different promoters. As reference, a plasmid encoding mIL12 under the control of the P7.5 promoter was used. Cytosine β-D-arabinofuranoside (AraC), an inhibitor of DNA replication, was added to the cells to block intermediate and late MVA gene expression. This allows analysis of only early promoters activity in the absence of viral functions supporting late expression (Chakrabarti S., Sisler J. R., Moss B. Compact, synthetic, vaccinia virus early/late promoter for protein expression. Biotechniques. 1997; 23:1094-1097). All the tested SynE promoters drive early mIL12 expression at higher levels (≥2-fold) compared to P7.5 (FIG. 12c).

1-15. (canceled)

16. A recombinant Orthopox viral vector comprising, in operable linkage:
a) a first promoter comprising:
(i) at least one viral early promoter element, wherein the viral early promoter element comprises the nucleic acid sequence AAN¹N²AN³TGAAN⁴N⁵N⁶N⁷N⁸A (SEQ ID NO:1), wherein each of N¹, N², N⁴, N⁵ and N⁶ is independently selected from A or T, N³ is selected from C, G or T, N⁷ is selected from C and A, and N⁸ is selected from A, C and T, and optionally at least one viral late promoter element; or
(ii) at least one viral late promoter element and at least three viral early promoter elements; and
b) a first nucleic acid sequence encoding at least one immunostimulatory protein.

17. The recombinant Orthopox viral vector of claim 16, wherein the Orthopox viral vector is a complete virus particle of the species selected from the group consisting of Orthopoxvirus variola, Orthopoxvirus vaccinia, Orthopoxvirus simiae, Orthopoxvirus bovis, Orthopoxvirus muris, Orthopoxvirus cameli, Raccoonpox virus, and Taterapox virus.

18. The recombinant Orthopox viral vector of claim 16, wherein the at least one immunostimulatory protein is a pro-inflammatory protein.

19. The recombinant Orthopox viral vector of claim 18, wherein the pro-inflammatory protein is IL-12.

20. The recombinant Orthopox viral vector of claim 16 further comprising a second nucleic acid sequence encoding one or more tumour antigens or an antigenic fragment thereof.

21. The recombinant Orthopox viral vector of claim 20, wherein the second nucleic acid sequence is operably linked to the first promoter or to a second promoter.

22. The recombinant Orthopox viral vector of claim 16, wherein each of the at least three viral early promoter elements is independently selected from the group consisting of:
(i) a Vaccinia Virus (VV) p7.5 early promoter element;
(ii) a viral early promoter element comprising or consisting of the nucleic acid sequence AAN¹N²AN³TGAAN⁴N⁵N⁶N⁷N⁸A (SEQ ID NO: 1), wherein each of N¹, N², N⁴, N⁵ and N⁶ is independently selected from A or T, N³ is selected from C, G or T, N⁷ is selected from C and A, and N⁸ is selected from A, C and T;
(iii) a viral early promoter element comprising the nucleic acid sequence AAN¹N²AN³TGAAN⁴N⁵N⁶N⁷N⁸AN⁹TCTAATTTATTGN¹⁰AN¹¹GG (SEQ ID NO: 2), wherein each of N¹, N², N⁴, N⁵, and N⁶ is independently selected from A or T, N³ is selected from C, G, or T, N⁷ is selected from C and A, N⁸ is selected from A, C and T, N⁹ is selected from G and T, N¹⁰ is selected from C and T and N¹¹ is selected from A and C;
(iv) a viral early promoter element comprising the nucleic acid sequence AAN¹N²AN³TGAAN⁴N⁵N⁶N⁷N⁸AGTCTAATTTATTGCACGG (SEQ ID NO: 3), wherein each of N¹, N², N⁴, N⁵ and N⁶ is independently selected from A or T, N³ is selected from C, G or T, N⁷ is selected from C and A, and N⁸ is selected from A, C, and T;
(v) a viral early promoter element with a nucleic acid sequence according to any one of SEQ ID NOs: 4 to 11 and SEQ ID NOs: 43 to 46 or any one of SEQ ID NOs: 4, 5, 43, 44, 45, and 46; and
(vi) an early promoter element comprising the nucleic acid sequence of SEQ ID NO: 4,
and/or
wherein each of the viral late promoter elements is independently selected from the group consisting of:
(i) Vaccinia Virus (VV) p7.5 late promoter element;
(ii) a late promoter element comprising the nucleic acid sequence TTTN¹N²N³N⁴N⁵N⁶N⁷N⁸N⁹TTTTTN¹⁰N¹¹N¹²N¹³N¹⁴N¹⁵N¹⁶ATAAATA, (SEQ ID NO: 41), wherein each of N¹ to N⁹ is independently selected from A, C, G, T or absent and N¹⁰ to N¹⁶ is independently selected from A, C, G, T or absent;
(iii) a viral late promoter element comprising the nucleic acid sequence TTTN¹N²N³N⁴N⁵N⁶N⁷N⁸N⁹TTTTTN¹⁰N¹¹N¹²N¹³N¹⁴N¹⁵N¹⁶ATAAATA, (SEQ ID NO: 42), wherein each of N¹ is selected from C or T, N² and N⁹ are in each case independently selected from A, G or T, N³, N⁴ and N⁸ are each independently selected from A or T, N⁵ is selected from G, T or absent, N⁶ is selected from A, T or absent, N⁷ is selected from A, G, T or absent, N¹⁰ is selected from C, G, and T, N¹¹ is selected from A, G, or T, N¹² is selected from A, and C, N¹³ is selected from T or absent, N¹⁴ is selected from G or absent, N¹⁵ is selected from A, C, and T and N¹⁶ is selected from A, C, and T;
(iv) a late promoter element with a nucleic acid sequence according to any one of SEQ ID NOs: 12 to 16; and
(v) a late promoter element comprising the nucleic acid sequence of SEQ ID NO: 12, and/or wherein each late and/or early promoter element is optionally connected by a nucleic acid linker having a length of 5, 6, 7, or 8 nucleotides, or
wherein the first promoter comprises a nucleic acid according to any one of SEQ ID NOs: 18 to 30.

23. A cell comprising the recombinant Orthopox viral vector of claim 16.

24. A composition, comprising:
a) a recombinant Orthopox viral vector comprising, in operable linkage:
a first promoter comprising
(i) at least one viral early promoter element, wherein the viral early promoter element comprises the nucleic acid sequence AAN¹N²AN³TGAAN⁴N⁵N⁶N⁷N⁸A (SEQ ID NO:1), wherein each of N¹, N², N⁴, N⁵, and N⁶ is independently selected from A or T, N³ is selected from C, G or T, N⁷ is selected from C and A, and N⁸ is selected from A, C and T, and optionally at least one viral late promoter element, or
(ii) at least one viral late promoter element and at least three viral early promoter elements, and
a first nucleic acid sequence encoding at least one immunostimulatory protein;
b) a pharmaceutically acceptable carrier; and optionally
c) a recombinant viral vector comprising a nucleic acid sequence encoding a checkpoint inhibitor, a nucleic acid encoding a checkpoint inhibitor, or a checkpoint inhibitor.

25. The composition of claim 24, wherein the checkpoint inhibitor is selected from the group consisting of a CTLA-4 inhibitor, a PD-1 inhibitor, and a PD-L1 inhibitor.

26. A method of treating cancer in a subject in need thereof, comprising:
a) administering to the subject a therapeutically effective amount of a composition comprising a recombinant Orthopox viral vector comprising, in operable linkage:
a first promoter comprising
(i) at least one viral early promoter element, wherein the viral early promoter element comprises the nucleic acid sequence AAN¹N²AN³TGAAN⁴N⁵N⁶N⁷N⁸A (SEQ ID NO: 1), wherein each of N¹, N², N⁴, N⁵, and N⁶ is independently selected from A or T, N³ is selected from C, G or T, N⁷ is selected from C and A, and N⁸ is selected from A, C, and T, and optionally at least one viral late promoter element, or
(ii) at least one viral late promoter element and at least three viral early promoter elements, and
a first nucleic acid sequence encoding at least one immunostimulatory protein,
a pharmaceutically acceptable carrier; and
b) at least one of reducing tumor growth and increasing overall survival in the subject.

27. The method of claim 26, wherein the cancer is breast cancer, small intestine cancer, stomach cancer, kidney cancer, bladder cancer, uterus cancer, ovarian cancer, testes cancer, lung cancer, colon cancer, prostate cancer, B cell lymphoma, Burkitt's lymphoma or Hodgkin's lymphoma.

28. The method of claim 26, wherein the composition is administered directly into a tumor by direct injection, or with a catheter prior to or during surgery.

29. The method of claim 26, wherein the therapeutically effective amount of the composition and a therapeutically effective amount of a recombinant viral vector comprising a nucleic acid sequence encoding a checkpoint inhibitor, a nucleic acid encoding a checkpoint inhibitor or a checkpoint inhibitor are administered to the subject simultaneously or sequentially.

30. The recombinant Orthopox viral vector of claim 17, wherein the complete virus particle is a Modified Vaccinia Ankara Virus (MVA).

31. The recombinant Orthopox viral vector of claim 19, wherein the IL-12 is a human single chain IL-12 (sc-hIL12) having an amino acid sequence of any one of SEQ ID NOs: 37 to 40.

32. The composition of claim 24, wherein the recombinant Orthopox viral vector is a complete Modified Vaccinia Ankara Virus (MVA) virus particle, and wherein the at least one immunostimulatory protein comprises a human single chain IL-12 (sc-hIL12) having an amino acid sequence of any one of SEQ ID NOs: 37 to 40.

33. The composition of claim 24, wherein each of the at least three viral early promoter elements is independently selected from the group consisting of:
(i) a Vaccinia Virus (VV) p7.5 early promoter element;
(ii) a viral early promoter element comprising the nucleic acid sequence AAN¹N²AN³TGAAN⁴N⁵N⁶N⁷N⁸A (SEQ ID NO: 1), wherein each of N¹, N², N⁴, N⁵, and N⁶ is independently selected from A or T, N³ is selected from C, G or T, N⁷ is selected from C and A, and N⁸ is selected from A, C and T;
(iii) a viral early promoter element comprising the nucleic acid sequence AAN¹N²AN³TGAAN⁴N⁵N⁶N⁷N⁸AN⁹TCTAATTTATTGN¹⁰AN¹¹GG (SEQ ID NO: 2), wherein each of N¹, N², N⁴, N⁵ and N⁶ is independently selected from A or T, N³ is selected from C, G or T, N⁷ is selected from C and A, N⁸ is selected from A, C and T, N⁹ is selected from G and T, N¹⁰ is selected from C and T and N¹¹ is selected from A and C;
(iv) a viral early promoter element comprising the nucleic acid sequence AAN¹N²AN³TGAAN⁴N⁵N⁶N⁷N⁸AGTCTAATTTATTGCACGG (SEQ ID NO: 3), wherein each of N¹, N², N⁴, N⁵ and N⁶ is independently selected from A or T, N³ is selected from C, G or T, N⁷ is selected from C and A, and N⁸ is selected from A, C, and T;
(v) an early promoter element with a nucleic acid sequence according to any one of SEQ ID NOs: 4 to 11 and SEQ ID NOs: 43 to 46 or any one of SEQ ID NOs: 4, 5, 43, 44, 45, and 46; and
(vi) an early promoter element comprising the nucleic acid sequence of SEQ ID NO: 4, and/or
wherein each of the viral late promoter elements is independently selected from the group consisting of:
(i) Vaccinia Virus (VV) p7.5 late promoter element;
(ii) a late promoter element comprising the nucleic acid sequence TTTN¹N²N³N⁴N⁵N⁶N⁷N⁸N⁹TTTTTN¹⁰N¹¹N¹²N¹³N¹⁴N¹⁵N¹⁶ATAAATA (SEQ ID NO: 41), wherein each of N¹ to N⁹ is independently selected from A, C, G, T, or absent and N¹⁰ to N¹⁶ is independently selected from A, C, G, T, or absent;
(iii) a viral late promoter element comprising the nucleic acid sequence TTTN¹N²N³N⁴N⁵N⁶N⁷N⁸N⁹TTTTTN¹⁰N¹¹N¹²N¹³N¹⁴N¹⁵N¹⁶ATAAATA (SEQ ID NO: 42), wherein each of N¹ is selected from C or T, N² and N⁹ are in each case independently selected from A, G or T, N³, N⁴ and N⁸ are each independently selected from A or T, N⁵ is selected from G, T or absent, N⁶ is selected from A, T or absent, N⁷ is selected from A, G, T or absent, N¹⁰ is selected from C, G, and T, N¹¹ is selected from A, G or T, N¹² is selected from A, and C, N¹³ is selected from T or absent, N¹⁴ is selected from G or absent, N¹⁵ is selected from A, C, and T and N¹⁶ is selected from A, C, and T;
(iv) a late promoter element with a nucleic acid sequence according to any one of SEQ ID NOs: 12 to 16; and
(v) a late promoter element comprising the nucleic acid sequence of SEQ ID NO: 12, and/or
wherein each late and/or early promoter element is optionally connected by a nucleic acid linker having a length of 5, 6, 7, or 8 nucleotides, or
wherein the first promoter comprises a nucleic acid according to one of SEQ ID NOs: 18 to 30.

34. The composition of claim 25, wherein the checkpoint inhibitor is a PD-1 inhibitor comprising one or more of Nivolumab, Atezolizumab, Pembrolizumab, Cemiplimab, Durvalumab, and Avelumab.

35. The method of claim 26 further comprising administering to the subject a therapeutically effective amount of a recombinant viral vector comprising a nucleic acid sequence encoding a checkpoint inhibitor, a nucleic acid encoding a checkpoint inhibitor, or a checkpoint inhibitor.


SEQUENCE LISTING (46 sequences)

SEQ ID NO: 1 - skipped

SEQ ID NO: 2 - DNA, 34 bp, synthetic construct
  misc_feature 1..34: note = Consensus early promoter element
  misc_difference 3: note = N is A or T, preferably A
  misc_difference 4: note = N is A or T, preferably A
  misc_difference 6: note = N is C, G or T, preferably T
  misc_difference 11: note = N is A or T, preferably A
  misc_difference 12: note = N is A or T, preferably A
  misc_difference 13: note = N is A or T, preferably A
  misc_difference 14: note = N is C and A, preferably C
  misc_difference 15: note = N is A, C and T, preferably T
  misc_difference 17: note = N is G and T, preferably G
  misc_difference 30: note = N is C and T, preferably C
  misc_difference 32: note = N is A and C, preferably C
  source 1..34: mol_type = other DNA
aannantgaannnnnantctaatttattgnangg

SEQ ID NO: 3 - DNA, 34 bp, synthetic construct
  misc_feature 1..34: note = Consensus early promoter element
  misc_difference 3: note = N is A or T, preferably A
  misc_difference 4: note = N is A or T, preferably A
  misc_difference 6: note = N is C, G or T, preferably T
  misc_difference 11: note = N is A or T, preferably A
  misc_difference 12: note = N is A or T, preferably A
  misc_difference 13: note = N is A or T, preferably A
  misc_difference 14: note = N is C and A, preferably C
  misc_difference 15: note = N is A, C and T, preferably T
  source 1..34: mol_type = other DNA
aannantgaannnnnagtctaatttattgcacgg

SEQ ID NO: 4 - DNA, 34 bp, synthetic construct
  misc_feature 1..34: note = Artificial Early Promoter Element
  source 1..34: mol_type = other DNA
aaaaattgaaaaactagtctaatttattgcacgg

SEQ ID NO: 5 - DNA, 34 bp, synthetic construct
  misc_feature 1..34: note = Artificial Early Promoter Element
  source 1..34: mol_type = other DNA
aaaaattgaaaataaagtctaatttattgcacgg

SEQ ID NO: 6 - DNA, 34 bp, synthetic construct
  misc_feature 1..34: note = Artificial Early Promoter Element
  source 1..34: mol_type = other DNA
aaaaagtgaaaaacaagtctaatttattgcacgg

SEQ ID NO: 7 - DNA, 34 bp, synthetic construct
  misc_feature 1..34: note = Artificial Early Promoter Element
  source 1..34: mol_type = other DNA
aaaaagtgaaaatatagtctaatttattgcacgg

SEQ ID NO: 8 - DNA, 34 bp, synthetic construct
  misc_feature 1..34: note = Artificial Early Promoter Element
  source 1..34: mol_type = other DNA
aaaaattgaaaaactattctaatttattgcacgg

SEQ ID NO: 9 - DNA, 34 bp, synthetic construct
  misc_feature 1..34: note = Artificial Early Promoter Element
  source 1..34: mol_type = other DNA
aaaaattgaaaaactagtctaatttattgtaagg

SEQ ID NO: 10 - DNA, 34 bp, synthetic construct
  misc_feature 1..34: note = Artificial Early Promoter Element
  source 1..34: mol_type = other DNA
aattactgaattaatagtctaatttattgcacgg

SEQ ID NO: 11 - DNA, 34 bp, synthetic construct
  misc_feature 1..34: note = Artificial Early Promoter Element
  source 1..34: mol_type = other DNA
aaatattgaaaaaacagtctaatttattgcacgg

SEQ ID NO: 12 - DNA, 49 bp, synthetic construct
  misc_feature 1..49: note = Artificial Late Promoter Element
  source 1..49: mol_type = other DNA
aattggatcagcttttttttttttttttttggcatataaataaggtcga

SEQ ID NO: 13 - DNA, 49 bp, synthetic construct
  misc_feature 1..49: note = Artificial Late Promoter Element
  source 1..49: mol_type = other DNA
aaacccacccgctttttatagtaagtttttcacccataaataataaata

SEQ ID NO: 14 - DNA, 35 bp, synthetic construct
  misc_feature 1..35: note = Artificial Late Promoter Element
  source 1..35: mol_type = other DNA
gtttaaacgttttgaaaatttttttataataaata

SEQ ID NO: 15 - DNA, 551 bp, synthetic construct
  misc_feature 1..551: note = Artificial Late Promoter Element
  source 1..551: mol_type = other DNA
aataaatatcgaagttgaagatgatgatgatgatgatgatgatgatgatgatgatgatgatgatgatgatgatgatgatgatgatgatgatgatgatgatgatgtcatagacgatgatgattataatccaaaacccactccgataccggagcctcaccctagaccaccgtttcccagacatgaatatcataagaggccgaaagttcttcctgtagaagaacctgatcctgtcaaaaaagacgcggatcgtataagacttgataatcatatattaaacacattggatcataatcttaattccatcggacactattgttgtgatacagcagcagttgataggttagaacatcacattgaaacattgggacaatatgcagtaatactagcaagaaagataaatatgcaaacattactgttcccatggccattacctactgtccatccacatgcgatagatggtagtattccgccacatgggagatctacgatcttataattacacgattgtagttaagttttgaataaaatttttttataataaatagaggtca

SEQ ID NO: 16 - DNA, 54 bp, synthetic construct
  misc_feature 1..54: note = Artificial Late Promoter Element
  source 1..54: mol_type = other DNA
taaaaatatagtagaatttcattttgtttttttctatgctataaataaattctc

SEQ ID NO: 17 - DNA, 141 bp, Vaccinia virus
  source 1..141: mol_type = other DNA
gatcactaattccaaacccacccgctttttatagtaagtttttcacccataaataataaatacaataattaatttctcgtaaaagtagaaaatatattctaatttattgcacggtaaggaagtagaatcataaagaacagt

SEQ ID NO: 18 - DNA, 214 bp, synthetic construct
  misc_feature 1..214: note = Assembled Synthetic (SynE) promoter 1
  source 1..214: mol_type = other DNA
aattggatcagcttttttttttttttttttggcatataaataaggtcgaagcttaaaaattgaaaaactagtctaatttattgcacggtctcgtaaaaattgaaaaactagtctaatttattgcacggtctcgtaaaaattgaaaaactagtctaatttattgcacggtctcgtaaaaattgaaaaactagtctaatttattgcacggtctgga

SEQ ID NO: 19 - DNA, 160 bp, synthetic construct
  misc_feature 1..160: note = Assembled Synthetic (SynE) promoter 2
  source 1..160: mol_type = other DNA
aaaaattgaaaaactagtctaatttattgcacggtctcgtaaaaattgaaaaactagtctaatttattgcacggtctcgtaaaaattgaaaaactagtctaatttattgcacggtctcgtaaaaattgaaaaactagtctaatttattgcacggtctgga

SEQ ID NO: 20 - DNA, 215 bp, synthetic construct
  misc_feature 1..215: note = Assembled Synthetic (SynE) promoter 3
  source 1..215: mol_type = other DNA
aaacccacccgctttttatagtaagtttttcacccataaataataaatatccggtaaaaagtgaaaaacaagtctaatttattgcacggtccggtaaaaagtgaaaaacaagtctaatttattgcacggtccggtaaaaagtgaaaaacaagtctaatttattgcacggtccggtaaaaagtgaaaaacaagtctaatttattgcacggtccgga

SEQ ID NO: 21 - DNA, 215 bp, synthetic construct
  misc_feature 1..215: note = Assembled Synthetic (SynE) promoter 4
  source 1..215: mol_type = other DNA
aattggatcagcttttttttttttttttttggcatataaataaggtcgatccggtaaaaattgaaaaactagtctaatttattgtaaggtccggtaaaaattgaaaaactagtctaatttattgtaaggtccggtaaaaattgaaaaactagtctaatttattgtaaggtccggtaaaaattgaaaaactagtctaatttattgtaaggtccgga

SEQ ID NO: 22 - DNA, 717 bp, synthetic construct
  misc_feature 1..717: note = Assembled Synthetic (SynE) promoter 5
  source 1..717: mol_type = other DNA
aataaatatcgaagttgaagatgatgatgatgatgatgatgatgatgatgatgatgatgatgatgatgatgatgatgatgatgatgatgatgatgatgatgatgtcatagacgatgatgattataatccaaaacccactccgataccggagcctcaccctagaccaccgtttcccagacatgaatatcataagaggccgaaagttcttcctgtagaagaacctgatcctgtcaaaaaagacgcggatcgtataagacttgataatcatatattaaacacattggatcataatcttaattccatcggacactattgttgtgatacagcagcagttgataggttagaacatcacattgaaacattgggacaatatgcagtaatactagcaagaaagataaatatgcaaacattactgttcccatggccattacctactgtccatccacatgcgatagatggtagtattccgccacatgggagatctacgatcttataattacacgattgtagttaagttttgaataaaatttttttataataaatagaggtcatctcgtaaaaattgaaaaactagtctaatttattgcacggtctcgtaaaaattgaaaaactagtctaatttattgcacggtctcgtaaaaattgaaaaactagtctaatttattgcacggtctcgtaaaaattgaaaaactagtctaatttattgcacggtctgga

SEQ ID NO: 23 - DNA, 207 bp, synthetic construct
  misc_feature 1..207: note = Assembled Synthetic (SynE) promoter 6
  source 1..207: mol_type = other DNA
acgcgtgtttaaacgttttgaaaatttttttataataaatatccggtaaaaattgaaaaactagtctaatttattgcacggtccggtaaaaattgaaaaactagtctaatttattgcacggtccggtaaaaattgaaaaactagtctaatttattgcacggtccggtaaaaattgaaaaactagtctaatttattgcacggtccgga

SEQ ID NO: 24 - DNA, 221 bp, synthetic construct
  misc_feature 1..221: note = Assembled Synthetic (SynE) promoter 7
  source 1..221: mol_type = other DNA
ataaaaatatagtagaatttcattttgtttttttctatgctataaataaattctcataggaaaaaattgaaaataaagtctaatttattgcacggtctcgtaaaaattgaaaataaagtctaatttattgcacggtctcgtaaaaattgaaaataaagtctaatttattgcacggtctcgtaaaaattgaaaataaagtctaatttattgcacggtctgga

SEQ ID NO: 25 - DNA, 214 bp, synthetic construct
  misc_feature 1..214: note = Assembled Synthetic (SynE) promoter 8
  source 1..214: mol_type = other DNA
aattggatcagcttttttttttttttttttggcatataaataaggtcgaagcttaaaaattgaaaataaagtctaatttattgcacggtctcgtaaaaagtgaaaatatagtctaatttattgcacggtctcgtaaaaagtgaaaaacaagtctaatttattgcacggtctcgtaaaaattgaaaaactagtctaatttattgtaaggtctgga

SEQ ID NO: 26 - DNA, 221 bp, synthetic construct
  misc_feature 1..221: note = Assembled Synthetic (SynE) promoter 9
  source 1..221: mol_type = other DNA
ataaaaatatagtagaatttcattttgtttttttctatgctataaataaattctcataggaaaaaattgaaaaactagtctaatttattgcacggtctcgtaaaaattgaaaataaagtctaatttattgcacggtctcgtaaaaagtgaaaaacaagtctaatttattgcacggtctcgtaaaaagtgaaaatatagtctaatttattgcacggtctgga

SEQ ID NO: 27 - DNA, 214 bp, synthetic construct
  misc_feature 1..214: note = Assembled Synthetic (SynE) promoter 10
  source 1..214: mol_type = other DNA
aattggatcagcttttttttttttttttttggcatataaataaggtcgaagcttaaaaattgaaaataaagtctaatttattgcacggtctcgtaaaaagtgaaaatatagtctaatttattgcacggtctcgtaaaaagtgaaaaacaagtctaatttattgcacggtctcgtaaaaattgaaaaactagtctaatttattgcacggtctgga

SEQ ID NO: 28 - DNA, 214 bp, synthetic construct
  misc_feature 1..214: note = Assembled Synthetic (SynE) promoter 11
  source 1..214: mol_type = other DNA
aattggatcagcttttttttttttttttttggcatataaataaggtcgaagcttaattactgaattaatagtctaatttattgcacggtctcgtaattactgaattaatagtctaatttattgcacggtctcgtaattactgaattaatagtctaatttattgcacggtctcgtaattactgaattaatagtctaatttattgcacggtctgga

SEQ ID NO: 29 - DNA, 207 bp, synthetic construct
  misc_feature 1..207: note = Assembled Synthetic (SynE) promoter 12
  source 1..207: mol_type = other DNA
acgcgtgtttaaacgttttgaaaatttttttataataaatatccggtaaatattgaaaaaacagtctaatttattgcacggtccggtaaatattgaaaaaacagtctaatttattgcacggtccggtaaatattgaaaaaacagtctaatttattgcacggtccggtaaatattgaaaaaacagtctaatttattgcacggtccgga

SEQ ID NO: 30 - DNA, 214 bp, synthetic construct
  misc_feature 1..214: note = Assembled Synthetic (SynE) promoter 13
  source 1..214: mol_type = other DNA
aattggatcagcttttttttttttttttttggcatataaataaggtcgaagcttaaaaattgaaaataaagtctaatttattgcacggtctcgtaattactgaattaatagtctaatttattgcacggtctcgtaaaaagtgaaaaacaagtctaatttattgcacggtctcgtaaatattgaaaaaacagtctaatttattgcacggtctgga

SEQ ID NO: 31 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 32 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 33 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 34 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 35 - protein, 22 aa, Homo sapiens
MCHQQLVISWFSLVFLASPLVA

SEQ ID NO: 36 - protein, 22 aa, Homo sapiens
MCPARSLLLVATLVLLDHLSLA

SEQ ID NO: 37 - protein, 543 aa, synthetic construct
  REGION 1..543: note = murine single-chain IL12 with (G4S)3 linker
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 38 - protein, 537 aa, synthetic construct
  REGION 1..537: note = human single-chain IL12 with (G3S)3 linker and human IL12 p40 signal peptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 39 - protein, 541 aa, synthetic construct
  REGION 1..541: note = human single-chain IL12 with (G3S)4 linker and human IL12 p40 signal peptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 40 - protein, 537 aa, synthetic construct
  REGION 1..537: note = human single-chain IL12 with (G3S)3 linker and human IL12 p35 signal peptide
MCPARSLLLVATLVLLDHLSLAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 41 - DNA, 31 bp, synthetic construct
  misc_feature 1..31: note = Late Promoter Consensus Sequence
  misc_difference 4: note = N is A, C, G, T or absent, preferably T
  misc_difference 5: note = N is A, C, G, T or absent, preferably T
  misc_difference 6: note = N is A, C, G, T or absent, preferably T
  misc_difference 7: note = N is A, C, G, T or absent, preferably T
  misc_difference 8: note = N is A, C, G, T or absent, preferably T
  misc_difference 9: note = N is A, C, G, T or absent, preferably T
  misc_difference 10: note = N is A, C, G, T or absent, preferably T
  misc_difference 11: note = N is A, C, G, T or absent, preferably T
  misc_difference 12: note = N is A, C, G, T or absent, preferably T
  misc_difference 18: note = N is A, C, G, T or absent, preferably G
  misc_difference 19: note = N is A, C, G, T or absent, preferably G
  misc_difference 20: note = N is A, C, G, T or absent, preferably C
  misc_difference 21: note = N is A, C, G, T or absent, preferably absent
  misc_difference 22: note = N is A, C, G, T or absent, preferably absent
  misc_difference 23: note = N is A, C, G, T or absent, preferably A
  misc_difference 24: note = N is A, C, G, T or absent, preferably T
  source 1..31: mol_type = other DNA
tttnnnnnnnnntttttnnnnnnnataaata

SEQ ID NO: 42 - DNA, 31 bp, synthetic construct; identical to SEQ ID NO: 41
  misc_feature 1..31: note = Late Promoter Consensus Sequence
  misc_difference 4: note = N is C or T, preferably T
  misc_difference 5: note = N is A, G or T, preferably T
  misc_difference 6: note = N is A or T, preferably T
  misc_difference 7: note = N is A or T, preferably T
  misc_difference 8: note = N is G, T or absent, preferably T
  misc_difference 9: note = N is A, T or absent, (preferably T
  misc_difference 10: note = N is A, G, T or absent, preferably T
  misc_difference 11: note = N is A or T, preferably T
  misc_difference 12: note = N is A, G or T, preferably T
  misc_difference 18: note = N is C, G, T, preferably G
  misc_difference 19: note = N is A, G or T, preferably G
  misc_difference 20: note = N is A, or C, preferably C
  misc_difference 21: note = N is T or absent, preferably absent
  misc_difference 22: note = N is G or absent, preferably absent
  misc_difference 23: note = N is A, C, T, preferably A
  misc_difference 24: note = N is A, C, T, preferably T
  source 1..31: mol_type = other DNA

SEQ ID NO: 43 - DNA, 34 bp, unidentified
  source 1..34: mol_type = other DNA
aaaaattgaaaataaatacaaaggttcttgaggg

SEQ ID NO: 44 - DNA, 34 bp, unidentified
  source 1..34: mol_type = other DNA
aattactgaattaataatataaaattcccaatct

SEQ ID NO: 45 - DNA, 34 bp, unidentified
  source 1..34: mol_type = other DNA
aaaaagtgaaaatatatatcattatattacaaag

SEQ ID NO: 46 - DNA, 34 bp, unidentified
  source 1..34: mol_type = other DNA
aaaaagtgaaaaacaatattatttttatcgttgg